(19) United States
(12) Patent Application Publication
(10) Pub. No.: US 2013/0289385 A1
(43) Pub. Date: Oct. 31, 2013

(54) DEEP BRAIN STIMULATION OF MEMORY CIRCUITS IN ALZHEIMER'S DISEASE

(71) Applicants: Andres Lozano, Toronto (CA); J. Christopher Flaherty, Auburndale, FL (US)

(72) Inventors: Andres Lozano, Toronto (CA); J. Christopher Flaherty, Auburndale, FL (US)

(73) Assignee: Functional Neuromodulation, Inc., Toronto (CA)

(21) Appl. No.: 13/655,652

(22) Filed: Oct. 19, 2012

Related U.S. Application Data
(63) Continuation-in-part of application No. PCT/US2011/033101, filed on Apr. 19, 2011.
(60) Provisional application No. 61/325,729, filed on Apr. 19, 2010.

(51) Int. Cl. A61K 51/04; A61K 49/00; A61B 5/055; A61K 49/06

(52) U.S. Cl. CPC A61K 51/0491; A61K 49/06; A61K 49/00; A61B 5/055
USPC 600/411; 424/9.3; 424/9.1; 424/1.73; 600/421

(57) ABSTRACT

Methods and apparatus for screening patients prior to deep brain stimulation to treat cognitive function are provided. One or more patient parameters are processed to produce results. A comparison of the results to a threshold indicates the applicability of the deep brain stimulation therapy.

### CROSS REFERENCE TO RELATED APPLICATIONS

This application is a continuation-in-part of PCT Application No. PCT/US2011/033101 (Attorney Docket No. 41551-703.601), filed on Apr. 19, 2011, which claims the benefit of Provisional Application No. 61/325,729 (Attorney Docket No. 41551-703.101), filed Apr. 19, 2010, the contents of which are incorporated herein by reference in their entirety.

### BACKGROUND OF THE INVENTION

### 1. Field of the Invention

The present invention relates generally to treatment of Alzheimer's disease. In particular, the present invention provides deep brain stimulation to increase memory, reduce memory loss, and maintain level of memory.

Cognitive disorders are a common type of neurological disorders. For example, dementia is a form of impaired cognition caused by brain dysfunction. The hallmark of most forms of dementia is the disruption of memory performance. Among the several conditions labeled as dementia, the most common are Alzheimer's disease and mild cognitive impairment (MCI), which is a pre-clinical form of Alzheimer's disease. MCI is an intermediate state between normal aging and dementia and is characterized by acquired cognitive deficits, without significant decline in functional activities of daily living. Subjects with MCI and the initial phase of Alzheimer's disease originally present with a predominant deficit in memory function. In more advanced stages of Alzheimer's disease, impairment in additional cognitive domains culminates with a significant decline in quality of life and the inability to perform usual daily activities.

Alzheimer's disease is one of the most common cognitive disorders in humans and has an exponentially increasing incidence. Although the defining characteristic of Alzheimer's disease is cognitive impairment, it is often accompanied by mood and behavioral symptoms such as depression, anxiety, irritability, inappropriate behavior, sleep disturbance, psychosis, and agitation. Neuro-imaging and genetic testing have aided in the identification of individuals at increased risk for dementia. However, the measurement of change in cognitive and functional status in, for example, MCI remains challenging because it requires instruments that are more sensitive and specific than those considered adequate for research in dementia. Accordingly, no treatment exists that adequately prevents or cures Alzheimer's disease or MCI.

Alzheimer's disease and MCI are already a public health problem of enormous proportions. It is estimated that 5 million people currently suffer with Alzheimer's disease in the United States. This figure is likely underestimated due to the high number of unrecognized and undiagnosed patients in the community. By the year 2050, Alzheimer's is projected to affect 14 million people. Moreover, because the prevalence of Alzheimer's disease doubles every 5 years after age 65, the impact of the disease on society tends to increase with the growth of the elderly population. The annual cost in the United States of Alzheimer's Disease alone is approximately $100 billion.

There is currently no effective treatment for the memory loss and other cognitive deficits presented by patients with dementia, particularly Alzheimer's disease. Treating Alzheimer's disease tends to be more challenging than other neurological disorders because Alzheimer's largely affects a geriatric population. Oral medications including Acetylcholinesterase inhibitors and cholinergic agents are the mainstay treatment for this condition. Nevertheless, the outcome with these agents is modest and tends to decline as the disease progresses. Other agents, such as nonsteroidal anti-inflammatory drugs, corticosteroids, COX-2 inhibitors, estrogen, and antioxidants, have also been tried with poor results. Neurotrophic factors (molecules that increase survival and growth of neurons in laboratory experiments) have been recently used clinically for Alzheimer's disease. Because these agents are proteins, they are inactive with oral administration and cannot cross the blood-brain barrier when administered systemically. When infused intraventricularly in three patients with Alzheimer's disease, nerve growth factor (NGF) increased cerebral nicotine binding. However, this compound had only modest clinical effects and was associated with back pain and weight loss that were reversible with the cessation of treatment.

Alternative routes of neurotrophic factor administration are currently being studied. Gene therapy and small neurotrophic molecules that can penetrate the blood-brain barrier (AIT-082) are possible methods for drug delivery. Moreover, treatment strategies against beta-amyloid protein accumulation and plaque formation including immunotherapies with vaccines are other possible methods. However, clinical data is still lacking for any of these alternative methods for treating cognitive disorders.

Most aspects of memory function involve temporal lobe structures Amnesic syndromes have been described after the disruption of the hippocampus, amygdala, formix, mammilary bodies, anterior nucleus of the thalamus, rhinal cortex, parahippocampal cortex, and temporal neocortex. These structures are mainly involved with the so-called declarative memory, which comprises the memory for facts, events, spatial location, recognition of forms, significance of data processed, among others. However, no interventions within the temporal lobe have been successful in improving memory function.

The hippocampus also has been found to play a crucial role in learning and memory. Lesions of the hippocampus in rodents, primates and man have been found to impair the process of memory acquisition and its persistence. In addition, the hippocampus receives strong inputs from nuclei in the basal forebrain, including the septal nuclei, the diagonal band of Broca and the nucleus basalis of Meynert and lesions in these structures also impair learning and memory. Dysfunction or pathological changes in these circuits may contribute to memory and learning deficits in a variety of circumstances including old age and Alzheimer's disease. The finding of pathological changes in these structures (including synaptic and neuronal loss, senile plaques and neurofibrillary tangles) is characteristic of both age related and Alzheimer's type memory and learning dysfunction. Since septohippocampal lesions affect new learning to a greater extent than established memories, these structures appear to play an essential facilitory role in the establishment and consolidation of memory. Again, however, no interventions within the hippocampus or related structures have been successful in improving memory function.

It is therefore desirable to provide a technique for preventing or treating cognitive disorders such as Alzheimer's disease and/or, more broadly, to improve cognitive function in patients.

### BRIEF SUMMARY OF THE INVENTION

Developing an effective means to treat or reduce the effects of Alzheimer's disease is in great need. Therefore, provided herein are systems, methods and devices to treat Alzheimer's disease. Specifically described are methods to screen patients for deep brain stimulation therapy; methods to implant a deep brain stimulator into a patient; methods to optimize brain stimulation parameters for a patient; and devices and systems to treat an Alzheimer's patient.

According to a first aspect of the invention, a method of screening patients prior to deep brain stimulation to treat cognitive function is provided. At least one patient parameter is measured generating a first result. The first result is compared to a first threshold, and the patient is identified as a candidate for deep brain stimulation therapy based on this comparison. In some embodiments, the cognitive function treated comprises a memory function. The patient has typically been diagnosed with probable Alzheimer's disease, such as a diagnosis within the past two years. In some embodiments, the patient has been diagnosed with a disease or disorder selected from the group consisting of: a genetic form of Alzheimer's disease; mild cognitive impairment; hippocampal damage such as hippocampal damage due to Alzheimer's disease, anoxia, epilepsy and/or depression; a structural brain abnormality such as a tumor, an infarction, and/or an intracranial hematoma; and combinations of these. The patient is typically forty to eighty years old, such as a patient between fifty-five and eighty years old, and does not have any pre-existing structural brain abnormalities such as a tumor, an infarction, or an intracranial hematoma. In some embodiments, the patient has reduced integrity of white matter tracts innervating limbic structures such as the formix, such as can be determined using fractional anisotropy maps created using diffusion tensor imaging.

The deep brain stimulation may be applied to treat memory impairment; improve memory function; treat cognitive function loss; reverse synaptic loss; improve cognitive function; reduce degradation of cognitive function; promote neurogenesis in the hippocampus; drive neurotrophin expression; regulate biomarkers related to Alzheimer's disease such as abeta, tau and/or phosphor tau; increase neurotransmitter release such as acetylcholine; regulate BDNF expression; improve glucose utilization such as glucose utilization in the temporal lobe; and combinations of these.

The patient parameters measured are typically selected from the group consisting of: Mini-Mental State Examination (MMSE) level; Alzheimer's Disease Assessment Scale-Cognitive Subscale level; Clinical Dementia Rating-Sum of Boxes (CDR) level; Alzheimer's Disease Study Consortium—Activities of Daily Living level; Clinicians Interview-Based Impression of Change Plus Caregiver Input (CIBIC-plus) level; Neuropsychiatric Inventory (NPI) level; Electro Encephalography (EEG) signal, level or result of EEG signal analysis; PET image data or data analysis; fMRT image data or data analysis; MRI image data or data analysis such as hippocampal volume; diffusion tensor imaging data such as data including an assessment of the fractional anisotropy of the formix; and combinations of these.

In one embodiment, the first result comprises an MMSE score, the threshold comprises an MMSE value of 20, and the patient is defined as a candidate if the first result is greater than or equal to the first threshold. A second threshold may be included, such as an MMSE value of 29, and the patient is defined as a candidate if the first result is less than or equal to the second threshold. Alternatively or additionally, a patient parameter result may be an ADAS-cog score, such as when the patient is a candidate when the ADAS-cog/11 score is less than or equal to an ADAS-cog/11 score threshold of 24. In some embodiments, the patient can be identified as a candidate when the ADAS-cog/11 score is greater than or equal to 12 and less that or equal to 24. In some embodiments, the patient can be identified as a candidate when the results of an Item 1 ADAS-cog test is greater than or equal to 4.

In another embodiment, the first result comprises a CDR score and the threshold is a set of values including 0.5 and 1.0, and the patient is a candidate for deep brain stimulation therapy when the first result is included in these values.

In yet another embodiment, the first result comprises data obtained in a PET scan, such as data selected from the group consisting of: glucose utilization data; PET Pittsburgh compound B (PIB) data; and combinations of these.

After a patient is acceptably screened for deep brain stimulation therapy, a deep brain stimulator is implanted. In one embodiment, an MRI procedure is performed, such as to locate the formix target for stimulation. The deep brain stimulator may include a stimulating electrode, such that the electrode is positioned based on a formix target identified in an MRI image and/or to stimulate the Papez Circuit of the patient's brain. In one embodiment, the electrode is positioned 2 mm anterior and parallel to the vertical portion of the formix. Alternatively or additionally, one or more electrodes may be implanted such that the ventral-most contact is 2 mm above the dorsal surface of the optic tract, approximately 5 mm from the midline.

Deep brain stimulation energy may be delivered at a frequency between 20 and 200 Hz, typically 130 Hz. Voltage is typically applied between 1.0 and 10.0V, such as between 3.0 and 3.5V, and more typically less than 7.0V. Energy is delivered using pulse width modulation, such as with multiple pulses of 45-450 μsecond duration, such as with 90 μsecond pulses.

During or after deep brain stimulator implantation, one or more stimulation parameters may be optimized, such as parameters that avoid patient discomfort such as sweating; hallucinations; visual sensations; tingling; and combinations of these. EEG, magnetoencephalography or other neurophysiologic data may be recorded and analyzed to optimize stimulation parameters. A PET scan may be performed, such as to record blood flow and/or produce FDG data. The stimulator portion (e.g. one or more electrodes) may have implantation position confirmed such as via an MRI. An acute memory test can be performed, such as a test performed at varying stimulation settings. The acute memory can comprise a patient recall of words and/or images, and one or more stimulation parameters may be optimized based on the results of the acute memory test.

Deep brain stimulation therapy may include the delivery of one or more drugs or other agents, such as a cholinesterase inhibitor. A confirmation of drug tolerance, and/or a titration of drug dose may be performed.

According to another aspect of the invention, a method of implanting a deep brain stimulator in a patient to treat cognitive function is provided. A patient imaging procedure is performed collecting at least one patient image. A deep brain stimulator, including at least one stimulation element, is implanted. The at least one stimulation element is positioned at a stimulation location that is based on the at least one patient image.

One or more imaging procedures or techniques can be used, such as imaging procedures selected from the group consisting of: MRI; functional MRI (fMRI) X-ray; ultrasound imaging; PET scanning; and combinations of these. Multiple imaging procedures can be performed at different times, such as imaging procedures performed more than a week apart. These two images may be compared to determine any change in brain size; brain shape; brain thickness; and combinations of these.

Numerous stimulation elements can be used singly or in combination with other stimulation elements. Typical stimulation elements include but are not limited to: electromagnetic elements such as electrodes and magnets; optical stimulation elements such as visible or infrared light sources; and chemical stimulation elements such as an element configured to deliver biologically active molecules, neurotransmitters and/or neurotrophic factors.

A calibration or titration procedure may be performed during or after deep brain stimulator implantation. The deep brain stimulation implantation may be halted, or the deep brain stimulator removed if already implanted, under certain conditions, such as inability to complete a calibration or titration procedure. Typically calibration or titration procedures may adjust parameters selected from the group consisting of: electromagnetic energy delivery such as voltage or current delivered; light delivery such as wavelength or magnitude of light delivered; chemical parameters such as concentration of chemical delivered or rate of chemical delivery; and combinations of these. Alternatively or additionally, stimulator removal may be prompted by encountering a particular patient condition or state such as chest pain; labored breathing; twitching; unacceptable EKG signal; and/or unacceptable EEG signal. Other undesired patient conditions include but are not limited to: unacceptable neurological level of paranoia; psychosis; anxiety; and/or confusion.

One or more stimulation elements may be repositioned, during or after deep brain stimulator implantation, such as to maximize or minimize a patient parameter. In one embodiment, a stimulation element is repositioned to maximize recalled memory or memories. Alternatively or additionally, a stimulation element may be repositioned to minimize one or more of: chest pain; labored breathing; twitching; unacceptable EKG signal; unacceptable EEG signal; or an adverse neurological condition such as an unacceptable level of paranoia, psychosis, anxiety, or confusion.

According to another aspect of the invention, a method of optimizing stimulation parameters of a deep brain stimulator implanted in a patient to treat cognitive function is provided. A deep brain stimulator comprises at least one stimulation element constructed and arranged to deliver stimulation energy comprises one or more stimulation parameters. After the deep brain stimulator is implanted, the stimulation parameters are optimized, such as during or after the implantation procedure. Stimulation parameters to be optimized typically include parameters selected from the group consisting of: voltage levels; current levels; frequency of delivery; duty cycle parameters such as on time; and combinations of these.

Optimization of stimulation parameters may be determined after a level of patient discomfort is achieved, such as discomfort including one or more of: sweating; hallucinations; visual sensation; and tingling. Stimulation parameters may be modified based on one or more of: EEG recordings; magnetoencephalography recordings; or other patient physiologic recording. Stimulation parameters may be modified after analysis of a PET scan, such as a measurement of blood flow and/or fluorodeoxyglucose (FDG) data.

In other aspects, the present invention comprises:

A method for implanting a deep brain stimulator in a patient to treat cognitive function, said method comprising: performing a patient imaging procedure and collecting at least one patient image; implanting a deep brain stimulator, said stimulator comprising at least one stimulation element; wherein the at least one stimulation element is positioned at a stimulation location that is based on the at least one patient image.

A method wherein the patient imaging procedure is an MRI procedure.

A method wherein the at least one stimulation element is implanted said stimulator relative to a formix target identified on the at least one patient image.

A method wherein the patient imaging procedure further comprises performing a second imaging procedure selected form the group consisting of: x-ray; ultrasound imaging; fMRI; PET; and combinations thereof.

A method further comprising performing a second imaging procedure at a date earlier than the MRI procedure.

A method further comprising performing a comparative analysis of the MRI procedure and the second imaging procedure.

A method wherein the analysis compares one or more of: brain size; brain shape; or brain thickness.

A method wherein the patient imaging procedure is selected form the group consisting of: x-ray; ultrasound imaging; fMRI; PET; and combinations of these.

A method wherein the stimulation element is positioned based on a visual analysis of the at least one patient image.

A method wherein the stimulation element is positioned based on a computational analysis of the at least one patient image.

A method wherein said computational analysis comprises a mathematical analysis.

A method wherein said stimulation location is within the Papez Circuit of the patient's brain.

A method wherein said stimulation location is approximately 2 mm anterior and parallel to the vertical portion of the formix within the hypothalamus.

A method wherein the stimulating element comprises at least two electrodes.

A method wherein the at least one stimulating element comprises one or more electrodes, and wherein the ventral most electrode is positioned 2 mm above the dorsal surface of the optic tract, approximately 5 mm from the midline.

A method wherein the at least one stimulation element comprises an electrical stimulation element.

A method wherein the at least one stimulation element comprises an electrode.

A method wherein the at least one stimulation element comprises a magnet.

A method wherein the at least one stimulation element comprises an optical stimulation element.

A method wherein the optical stimulation element is selected from the group consisting of: a visible light stimulation element; an infrared light stimulation element; and combinations of these.

A method wherein the at least one stimulation element comprises a chemical stimulation element.

A method wherein the chemical stimulation element is constructed and arranged to deliver one or more of: biologically active molecules; neurotransmitters; neurotrophic factors.

A method wherein the at least one stimulation element comprises an electrode and a second stimulation element.

A method wherein the second stimulation element comprises a second electrode.

A method wherein the second stimulation element is selected from the group consisting of: a magnetic stimulation element; an optical stimulation element; a chemical stimulation element; and combinations of these.

A method further comprising explanting the deep brain stimulator from the patient.

A method wherein the deep brain stimulator is explanted due to an inability to calibrate or titrate said stimulator.

A method wherein the inability to calibrate or titrate comprises an inability to calibrate or titrate one or more of: electromagnetic energy delivery such as voltage or current delivered; light delivery such as wavelength or magnitude of light delivered; and chemical parameters such as concentration of chemical delivered or rate of chemical delivery.

A method wherein the deep brain stimulator is removed based on a patient condition encountered.

A method wherein the patient condition encountered is selected from the group consisting of: level chest pain; labored breathing; twitching; unacceptable EKG signal; unacceptable EEG signal; and combinations of these.

A method wherein the patient condition encountered is selected from the group consisting of: paranoia state; state of psychosis; state of anxiety; state of confusion; and combinations of these.

A method further comprising repositioning the at least one stimulation element.

A method wherein the repositioning is performed to maximize a patient parameter.

A method wherein the patient parameter comprises one or more of: a recalled memory and recalled memories.

A method wherein the repositioning is performed to minimize a patient parameter.

A method wherein the patient parameter comprises one or more of: chest pain; unacceptable EKG signal; unacceptable EEG signal; breathing state; or twitching.

A method wherein the patient parameter comprises one or more of: paranoia state; psychosis state; anxiety state; or confusion state.

A method further comprising delivering energy at a frequency between 20 and 200 Hz.

A method wherein the energy is delivered at a frequency of approximately 130 Hz.

A method further comprising delivering energy at a voltage between 1.0V and 10.0V.

A method wherein the energy is delivered at a voltage between approximately 3.0V and 3.5V.

A method wherein the energy is delivered at a voltage of less than or equal to 7.0V.

A method further comprising delivering energy in multiple pulses of 45-45 μsecond duration.

A method wherein the energy is delivered at an approximately 90 μsecond duration.

A method further comprising performing an optimization of stimulation parameters procedure.

A method wherein said optimization comprises determining a discomfort limit for the patient.

A method wherein the discomfort is selected from the group consisting of: sweating; hallucinations; visual sensations; tingling; and combinations of these.

A method wherein said optimization procedure is performed during the stimulator implantation surgery.

A method wherein said optimization procedure is performed after the stimulator implantation surgery.

A method further comprising recording EEG data, wherein said optimization procedure is based on said EEG data.

A method further comprising recording magnetoencephalography data, wherein said optimization procedure is based on said magnetoencephalography data.

A method further comprising performing a PET scan.

A method wherein data collected during the PET scan is selected from the group comprising: blood flow; FDG data; and combinations of these.

A method wherein said optimization procedure is based on said collected data.

A method further comprising confirming the placement of the electrode after surgery.

A method wherein said confirmation step is performed using MRI.

A method further comprising delivering a drug or other agent to the patient.

A method wherein the drug or other agent comprises at least one cholinesterase inhibitor.

A method further comprising assessing a patient tolerance to at least one drug.

A method wherein the at least one drug is delivered to the patient if the patient tolerance is within a clinically acceptable limit.

A method wherein the at least one drug is a cholinesterase inhibitor.

A method wherein the at least one drug is not delivered to the patient if the patient tolerance is not within a clinically acceptable limit.

A method wherein the patient has been diagnosed with Alzheimer's disease within the past two years.

A method wherein the patient has an age between forty and eighty years. A method wherein the patient has an age between fifty-five and eighty years.

A method wherein the patient has been taking cholinesterase inhibitor for at least six months.

A method wherein the patient has been diagnosed with a genetic form of Alzheimer's disease.

A method wherein the patient has been diagnosed as an Apo E4 allele carrier.

A method wherein the patient has been diagnosed with mild cognitive impairment.

A method wherein the patient has been diagnosed with hippocampal damage.

A method wherein the hippocampal damage is due to at least one of: anoxia, epilepsy and depression.

A method wherein the patient has not been diagnosed with a structural brain abnormality.

A method wherein the structural brain abnormality is selected from the group consisting of: a tumor; an infarction; an intracranial hematoma; and combinations of these.

A method wherein the deep brain stimulator treats cognitive function loss.

A method wherein the deep brain stimulator reverses synaptic loss.

A method wherein the deep brain stimulator improves cognitive function.

A method wherein the deep brain stimulator reduces degradation of cognitive function.

A method wherein the deep brain stimulator promotes neurogenesis in the hippocampus of the patient's brain.

A method wherein the deep brain stimulator drives neurotrophin expression.

A method wherein the deep brain stimulator regulates one or more biomarkers related to Alzheimer's disease.

A method wherein the one or more biomarkers are selected from the group consisting of: abeta; tau; phosphor tau; and combinations of these.

A method wherein the deep brain stimulator regulates BDNF expression.

A method wherein the deep brain stimulator improves glucose utilization in the temporal lobe, the parietal lobe or both lobes of the patient's brain.

A method further comprising: performing a patient screening procedure prior to implanting the deep brain stimulator, said patient screening procedure comprising: measuring at least one patent parameter to generate at least a first result; comparing the first result to a first threshold; identifying the patient as a candidate for deep brain stimulation therapy based on said comparison of the first result to the first threshold.

A method wherein the at least one patient parameter is selected from the group consisting of: Mini-Mental State Examination (MMSE) level; Alzheimer's Disease Assessment Scale-Cognitive Subscale level; Clinical Dementia Rating-Sum of Boxes (CDR) level; Alzheimer's Disease Study Consortium—Activities of Daily Living level; Clinicians Interview-Based Impression of Change Plus Caregiver Input (CIBIC-plus) level; Neuropsychiatric Inventory (NPI) level; Electro Encephalography (EEG) signal, level or result of EEG signal analysis; PET image data or data analysis; FMRI image data or data analysis; MRI image data or data analysis such as hippocampal volume; and combinations of these.

A method wherein the first result comprises an MMSE score, wherein the first threshold comprises an MMSE value of 20, and wherein the patient is a candidate for DBS if said first result is greater than or equal to the first threshold.

A method further comprising comparing said first result to a second threshold, said second threshold comprising an MMSE value of 29, wherein the patient is a candidate for DBS if said first result is less than or equal to said second threshold.

A method further comprising generating a second result and comparing said second result to a second threshold, said second result comprising an ADAS-cog score, said second threshold comprising an ADAS-cog/11 score value of 24, and wherein the patient is a candidate for DBS if said second result is less than or equal to said second threshold.

A method wherein the first result comprises an ADAS-cog score, wherein the first threshold comprises an ADAS-cog/11 value of 24, and wherein the patient is a candidate for DBS if said first result is less than or equal to the first threshold.

A method further comprising generating a second result and comparing said second result to a second threshold, said second result comprising an MMSE, said second threshold comprising an MMSE value of 20, and wherein the patient is a candidate for DBS if said second result is greater than or equal to said second threshold.

A method wherein the first result comprises a CDR score, wherein the first threshold comprises a set of values including 0.5 and 1.0, and wherein the patient is a candidate for DBS if said first result is included in the threshold set of values.

A method of optimizing the stimulation parameters of a deep brain stimulator implanted in a patient to treat cognitive function, said method comprising: implanting a deep brain stimulator, said stimulator comprising at least one stimulation element constructed and arranged to deliver stimulation energy comprising one or more stimulation parameters; optimizing the stimulation parameters.

A method wherein the stimulation parameters comprises voltage levels.

A method wherein the stimulation parameters comprises current levels.

A method wherein the stimulation parameters comprises frequency levels.

A method wherein the stimulation parameters comprises duty cycle proportions or periods.

A method wherein the stimulation parameters comprise duty cycle on time period.

A method wherein the optimizing comprises determining a patient discomfort level.

A method wherein patient discomfort comprises a condition selected form the group consisting of: sweating; hallucinations; visual sensations; tingling; and combinations of these.

A method wherein the optimizing is performed during the stimulator implantation surgery.

A method wherein the optimizing is performed after the stimulator implantation surgery.

A method further comprising recording EEG data, wherein said optimizing is based on said EEG data.

A method further comprising recording magnetoencephalography data, wherein said optimizing is based on said magnetoencephalography data.

A method further comprising performing a PET scan.

A method wherein data collected during the PET scan is selected from the group comprising: blood flow; FDG data; and combinations of these.

A method wherein said optimizing is based on said collected data.

A method further comprising delivering energy at a frequency between 20 and 200 Hz.

A method wherein the energy is delivered at a frequency of approximately 130 Hz.

A method further comprising delivering energy at a voltage between 1.0V and 10.0V.

A method wherein the energy is delivered at a voltage between approximately 3.0V and 3.5V.

A method wherein the energy is delivered at a voltage of less than or equal to 7.0V.

A method further comprising delivering energy in multiple pulses of 45 μsecond to 450 μsecond duration.

A method wherein the energy is delivered in multiple pulses of 90 μsecond duration.

A method further comprising confirming the placement of the electrode after surgery.

A method wherein said confirmation step is performed using MRI.

A method further comprising delivering a drug or other agent to the patient.

A method wherein the drug or other agent comprises at least one cholinesterase inhibitor.

A method further comprising assessing a patient tolerance to at least one drug.

A method wherein the at least one drug is delivered to the patient if the patient tolerance is within a clinically acceptable limit.

A method wherein the at least one drug is a cholinesterase inhibitor.

A method wherein the at least one drug is not delivered to the patient if the patient tolerance is not within a clinically acceptable limit.

A method wherein the patient has been diagnosed with Alzheimer's disease within the past two years.

A method wherein the patient has an age between forty and eighty years.

A method wherein the patient has been taking cholinesterase inhibitor for at least six months.

A method wherein the patient has been diagnosed with a genetic form of Alzheimer's disease.

A method wherein the patient has been diagnosed as an Apo E4 allele carrier.

A method wherein the patient has been diagnosed with mild cognitive impairment.

A method wherein the patient has been diagnosed with hippocampal damage.

A method wherein the hippocampal damage is due to at least one of: anoxia, epilepsy or depression.

A method wherein the patient has not been diagnosed with a structural brain abnormality.

A method wherein the structural brain abnormality is selected from the group consisting of: a tumor; an infarction; an intracranial hematoma; and combinations of these.

A method wherein the deep brain stimulator treats cognitive function loss.

A method wherein the deep brain stimulator reverses synaptic loss.

A method wherein the deep brain stimulator improves cognitive function.

A method wherein the deep brain stimulator reduces degradation of cognitive function.

A method wherein the deep brain stimulator promotes neurogenesis in the hippocampus of the patient's brain.

A method wherein the deep brain stimulator drives neurotrophin expression.

A method wherein the deep brain stimulator regulates one or more biomarkers related to Alzheimer's disease.

A method wherein the one or more biomarkers are selected from the group consisting of: abeta; tau; phosphor tau; and combinations of these.

A method wherein the deep brain stimulator regulates BDNF expression.

A method wherein the deep brain stimulator improves glucose utilization in the temporal lobe, the parietal lobe or both lobes of the patient's brain.

A method further comprising: performing a patient screening procedure prior to implanting the deep brain stimulator, said patient screening procedure comprising: measuring at least one patent parameter to generate at least a first result; comparing the first result to a first threshold; identifying the patient as a candidate for deep brain stimulation therapy based on said comparison of the first result to the first threshold.

A method wherein the at least one patient parameter is selected from the group consisting of: Mini-Mental State Examination (MMSE) level; Alzheimer's Disease Assessment Scale-Cognitive Subscale level; Clinical Dementia Rating-Sum of Boxes (CDR) level; Alzheimer's Disease Study Consortium—Activities of Daily Living level; Clinicians Interview-Based Impression of Change Plus Caregiver Input (CIBIC-plus) level; Neuropsychiatric Inventory (NPI) level; Electro Encephalography (EEG) signal, level or result of EEG signal analysis; PET image data or data analysis; FMRI image data or data analysis; MRI image data or data analysis such as hippocampal volume; and combinations of these.

A method wherein the first result comprises an MMSE score, wherein the first threshold comprises an MMSE value of 20, and wherein the patient is a candidate for DBS if said first result is greater than or equal to the first threshold.

A method further comprising comparing said first result to a second threshold, said second threshold comprising an MMSE value of 29, wherein the patient is a candidate for DBS if said first result is less than or equal to said second threshold.

A method further comprising a generating a second result and comparing said second result to a second threshold, said second result comprising an ADAS-cog score, said second threshold comprising an ADAS-cog/11 value of 24, and wherein the patient is a candidate for DBS if said second result is less than or equal to said second threshold.

A method wherein the first result comprises an ADAS-cog score, wherein the first threshold comprises an ADAS-cog/11 value of 24, and wherein the patient is a candidate for DBS if said first result is less than or equal to the first threshold.

A method further comprising a generating a second result and comparing said second result to a second threshold, said second result comprising an MMSE, said second threshold comprising an MMSE value of 20, and wherein the patient is a candidate for DBS if said second result is greater than or equal to said second threshold.

A method wherein the first result comprises a CDR score, wherein the first threshold comprises a set of values including 0.5 and 1.0, and wherein the patient is a candidate for DBS if said first result is included in the threshold set of values.

A method wherein the first result comprises data obtained in a PET scan.

A method wherein the data represents glucose utilization.

A method wherein the data is PET Pittsburgh compound B (PiB) data.

An apparatus for treating Alzheimer's disease as described in reference to the above figures.

### BRIEF DESCRIPTION OF THE DRAWINGS

The accompanying drawings, which are incorporated in and constitute a part of this specification, illustrate various embodiments of the present invention, and together with the description, serve to explain the principles of the invention. In the drawings:

FIG. 1A illustrates the position of a DBS electrode as shown on a sagittal magnetic resonance image (left) and its projection onto a stereotactic atlas 3.5 mm from the midline (right), consistent with the present invention;

FIG. 1B illustrates magnetic resonance images of 6 AD patients showing position of the formix/hypothalamic DBS electrodes in axial (top), coronal (middle) and sagittal (bottom) planes, consistent with the present invention;

FIG. 2A is a chart of individual MMSE scores in 6 patients representing the change between the period 11 months before DBS surgery as compared to the period 11 months after DBS surgery, consistent with the present invention;

FIG. 2B is a chart of the relationship between starting level of disability as assessed by MMSE score 1 month prior to surgery and change in ADAS-cog score at 12 months versus baseline, consistent with the present invention;

FIGS. 3A and 3B illustrate averaged standardized low-resolution electromagnetic tomography (sLORETA) graphics showing three-dimensional reconstruction during formix/hypothalamic stimulation, consistent with the present invention;

FIG. 4 illustrates averaged PET scans in 5 patients at baseline (1 month prior to DBS surgery) and after 1 or 12 months of continuous bilateral DBS of the formix/hypothalamus, consistent with the present invention;

FIG. 5 is a flow chart of a method of identifying a patient as a candidate for DBS therapy, consistent with the present invention;

FIG. 6 is a flow chart of a method of implanting a deep brain stimulator; consistent with the present invention;

FIG. 7 is a flow chart of a method of optimizing stimulation parameters of a deep brain stimulator, consistent with the present invention;

FIG. 8 is a schematic of a deep brain stimulator, consistent with the present invention.

### DETAILED DESCRIPTION OF THE INVENTION

Reference will now be made in detail to the present embodiments of the invention, examples of which are illustrated in the accompanying drawings. Wherever possible, the same reference numbers will be used throughout the drawings to refer to the same or like parts.

Provided herein are systems, methods and devices for treatment of Alzheimer's disease. Alzheimer's disease (AD) is characterized by both structural and functional impairment in the neural elements and circuits underlying cognitive and memory functions. Applicant has hypothesized that formix/hypothalamus deep brain stimulation (DBS) could modulate neurophysiological activity in these pathological circuits and possibly produce clinical benefits. Applicant has conducted numerous clinical experiments. In 6 patients with mild AD, DBS drove neural activity in the “memory circuit” including the entorhinal and hippocampal areas and activated the brain's “default mode network”. PET scans showed an early and striking reversal of the impaired glucose utilization in the temporal and parietal lobes that was maintained after 12 months of continuous stimulation. Evaluation of the Alzheimer's Disease Assessment Scale (ADAS) cognitive subscale (such as the 11 item ADAS-cog assessment also referred to as “ADAS-cog/11”) and the Mini Mental State Examination (MMSE) indicated slowing in the rate of cognitive decline in some patients. There were no serious adverse events.

Alzheimer's disease (AD) is characterized by a progressive disturbance in cognitive function with memory being particularly affected. Various pathological processes including the deposition of fibrillar forms of amyloid beta protein, neuronal degeneration, synaptic loss, defects in neurotransmission and disruption of neural network activity have been implicated as possible contributors to the dysfunction (reviewed in Querfurth and LaFerla 2010; Pal op and Mucke 2010; Sperling et al., 2010). Pathological studies in AD have shown that these disturbances can occur in widespread brain regions but with a predilection for involvement of neural circuits serving memory. Neuroimaging has played a critical role in identifying the topography of dysfunctional brain areas showing both morphological and volumetric structural changes predating the cognitive symptoms and tracking with disease severity (reviewed in Petersen and Jack, 2009). These studies characterizing the anatomical correlates of progression of the degenerative process have emphasized loss of cortical thickness and atrophy, particularly in the entorhinal cortex and the hippocampus (Risacher et al., 2009).

The structural abnormalities in AD are tightly coupled to functional disturbances. Regional reduction in glucose utilization in the temporal lobe and posterior cingulate area is a finding in positron emission and single photon emission computerized tomography in patients with AD early in the disease course, as well as in individuals at genetic risk (Smith et al, 1992, Minoshima, et al., 1997, Reiman et al., 1996). These regions of the brain also show a propensity for fibrillar amyloid deposition as visualized at autopsy and in vivo using radioligands such as the Pittsburgh compound B ([1 1C]-PiB) both in AD patients and non-demented older subjects (Mormino et al., 2009; Fripp et al., 2008). Recent evidence suggests amyloid pathology interferes with synaptic transmission and the normal activity of brain regions supporting various cognitive and memory functions (Palop and Mucke, 2009; Sperling et al., 2009). However the mechanism linking amyloid deposition and impaired functional deficits remains controversial. The pathophysiologic significance of the amyloid deposition on neuronal function is complex as suggested by the observation of amyloid deposition in a significant number of cognitively healthy older subjects (Buckner et al., 2005) and by post-mortem studies in AD patients treated with an amyloid vaccine that show disease progression, despite evidence of amyloid clearance from the brain (Holmes et al., 2008). The sensitivity of amyloid imaging to diagnosis, prognosis and longitudinal AD progression are improved considerably however, when amyloid imaging is combined with measures of neuronal dysfunction, such as cerebral glucose metabolism or brain volumes (Engler et al., 2006, Mormino et al., 2009, Jack et al, 2009).

While details of the mechanism remain unclear, there is general agreement that these molecular and structural abnormalities produce functional alterations of the brain areas they affect. The evidence of functional alterations in memory networks is seen not only in AD patients but also in the elderly (Andrews-Hanna et al., 2007; Sperling et al. 2010). Recent evidence show that both aged individuals and AD patients have defects in heteromodal interconnected cortical areas known collectively as the “default mode network” (Raichle et al., 2001; Grecius et al., 2004 and reviewed in Buckner et al, 2008). Normal individuals show correlated activity within the default network during resting states and deactivation of the network when performing many cognitive tasks. In contrast, older individuals, particularly those with accumulation of brain amyloid, lose the expected deactivation and toggling of the default network during cognitive tasks (Miller et al., 2008; Sperling et al., 2009). Defects in default mode network function as a consequence of amyloid deposition or through other mechanisms, may be responsible for some of the multimodal cognitive and behavioral deficits in AD (Buckner et al, 2008, Seely et al., 2009; Sperling 2010). A corollary of these findings is that malfunction in one diseased brain area interferes secondarily with the activity of others which may perhaps be less affected by molecular and structural pathology but whose function is nevertheless disrupted by virtue being linked in the network. This suggests that AD may not only be a degenerative disease but can also be considered as a system-level disorder affecting several integrated pathways linking select cortical and subcortical sites working in concert in serving aspects of memory and cognition. If this were true, then there would be interest in modulating the activity of these dysfunctional networks in an attempt to normalize their function. However, the extent to which these various functional abnormalities can be ameliorated or reversed over the long term by symptomatic drugs, manipulating levels of deleterious proteins or any other means is largely unknown.

Advances in neurosurgical techniques and the introduction of deep brain stimulation, have made possible the modulation of the activity of several brain circuits including pain circuits (Davis et al, 1998), motor circuits in patients with Parkinson's disease (reviewed in Lang and Lozano, 1998; Davis et al., 1997), essential tremor (Koller et al, 1997), dystonia (Vidailhet et al., 2005) and Huntington's disease (Moro et al., 2004), as well as circuits modulating mood, in patients with treatment resistant depression (Mayberg et al., 2005, Lozano et al., 2008). Interventions in these dysfunctional circuits can have local, trans-synaptic and remote effects (Davis et al., 1997; Mayberg et al., 2005), and in some cases can produce striking clinical improvements beyond what is achievable with medications.

Applicant has investigated the possibility of modulating memory circuitry activity in a patient with obesity using DBS of the formix and hypothalamus (Hamani et al., 2008). Applicant provoked reversible memory phenomena (retrieval of distinct autobiographical episodes) with acute high intensity stimulation. Source localization of the acute EEG effects showed activation in the hippocampal formation and the medial temporal lobe. These physiological changes were associated with acute and sustained improvements in memory, particularly those known to be dependent upon hippocampal-integrity, such as verbal recollection. These preliminary observations support the notion that the neural elements subserving certain memory functions are accessible in humans and that it is feasible to modulate their activity using electrical stimulation of the formix/hypothalamus.

The formix is a large axonal bundle that constitutes a major inflow and output pathway from the hippocampus and medial temporal lobe. In humans it is estimated to have 1.2 million axons (Powell et al., 1957) Importance of the formix to memory function is supported by the observation that lesions in the formix in experimental animals and humans are well known to produce memory deficits (Tsivilis et al., 2008; Wilson et al., 2008; Browning et al., 2009; Vann et al., 2009). Applicant hypothesized that it might be possible to use DBS of the formix to drive its activity and to modulate the circuits mediating memory function in patients with impairments in this domain. Applicant considered that patients with early or mild Alzheimer's disease would have both sufficient remaining structural integrity of these circuits and, given the unrelenting progressive nature of the impairment and the unfavorable natural course of the illness, a compelling reason to consider this experimental approach. Using the stimulation protocol used in Applicant's sentinel case as a launching point (Hamani et al., 2008), Applicant tested the hypothesis that stimulation in the formix/hypothalamus could alter activity in medial temporal memory circuits, providing a safe and potentially beneficial impact on memory in 6 patients with early Alzheimer's disease.

### Experimental Procedures—Patients

Six participants were recruited through the Memory Clinic at the Toronto Western Hospital for this pilot study. The criteria for inclusion were: (i) Men and women aged 40 to 80 years old, who (ii) satisfy the diagnostic criteria for probable AD (McKhann et al. 1984), (iii) have received the diagnosis of AD within the past 2 years, (iv) have a CDR of 0.5 or 1.0 (Morris 1993), and (v) score between 18 and 28 on the Mini Mental State Examination (MMSE; Folstein et al. 1975) and (vi) have been taking a stable dose of cholinesterase inhibitors for a minimum of 6 months. The exclusion criteria were: (i) pre-existing structural brain abnormalities (such as tumor, infarction, or intracranial hematoma), (ii) other neurologic or psychiatric diagnoses, or (iii) medical comorbidities that would preclude them from undergoing surgery.

### Experimental Procedures—Surgery

A Leksell stereotactic frame was applied to the patient's head under local anesthesia the morning of the procedure. Magnetic resonance brain imaging (MRI) was obtained. The right and left formix were readily seen on MRI images. The electrode target was chosen to lie 2 mm anterior and parallel to the vertical portion of the formix within the hypothalamus. The ventral most contact was 2 mm above the dorsal surface of the optic tract, approximately 5 mm from the midline. With the targets identified, deep brain stimulation electrodes (Medtronic model 3387; Medtronic, Minneapolis, Minn.) were implanted bilaterally with fluoroscopic guidance while the patient was awake. Once the electrodes were placed, stimulation was applied to survey for recollective experiences and adverse effects including distracting or unpleasant sensations (e.g. sweating, hallucinations, visual sensations, tingling, etc.). The electrodes were internalized and connected to an internal pulse generator (model Kinetra, Medtronic) implanted in the subcutaneous layer of the patient's chest while under general anesthetic. On the day following surgery, an MRI was obtained to confirm electrode placement. Patients were discharged 1 to 3 days following the operation with stimulators turned off.

### Experimental Procedures—Clinical Evaluation and Followup

Patients were seen 2 weeks after discharge from Hospital to have the stimulators turned on. Each contact was tested by fixing the frequency at 130 Hz and the pulse width at 90 microseconds and increasing the voltage from 1 to a maximum of 10 Volts. As in the operating room, high voltage settings, usually above 7 volts produced flushing, a sensation of warmth and increases in heart rate and blood pressure. Final voltage settings were below the threshold voltage for recollective experiences and mildly unpleasant sensations. All patients had chronic stimulation at 3.0 to 3.5 Volts with the frequency set at 130 Hertz and the pulse width at 90 microseconds. Stimulator settings and medications were kept constant for 12 months. Patients had neurological, neurosurgical and neuropsychological assessments at baseline and 1, 6 and 12 months following surgery.

The main outcome measure from the neuropsychological assessment was the Alzheimer's Disease Assessment Scale, Cognitive Subscale (ADAS-Cog/11; Rosen et al. 1984). This was chosen due to its widespread use in clinical dementia trials as well as algorithms to predict rate of decline in AD patients as a function of baseline scores (Stern et al., 1994; Ito et al., 2010). It includes components assessing declarative memory, orientation, praxis, and receptive and expressive language. Other measures included the MMSE (Folstein et al. 1975), the Clinical Dementia Rating, (CDR, Morris 1992), the Clinicians' Interview-Based Impression of Change—Plus Caregiver Input (CIBIC-Plus; Schneider et al. 1997), and the Quality of Life—Alzheimer Disease Scale (QOL-AD; Logsdon et al. 2002). MMSE data was available one year prior to the patients enrolling in the trial, so that the trajectory of clinical change for the year preceding and following DBS could be compared.

Experimental Procedures—Standardized Low-Resolution Electromagnetic Tomography (sLORETA)

sLORETA was used in the period of 6-12 months after insertion of the DBS electrodes to identify brain areas showing a focal change in activity in the electroencephalogram (EEG) in response to stimulation in all patients. For sLORETA, bipolar stimulation of the hypothalamus was conducted at 3 Hz with each electrode contact being investigated independently (130 Hz was not used because of associated high-frequency electrographic artifacts that preclude analysis with sLORETA) as previously described (Hamani et al., 2008). The intensities applied varied between 1 volt and 10 volts, and the pulse width was 450 microseconds. Five hundred consecutive stimuli were time-locked, and the evoked responses were averaged and compared with baseline electroencephalographic activity. sLORETA presents blurred images of statistically standardized current density distributions on a cortical grid of 6,239 voxels with accurate localization (Pascual-Marqui, 2002).

### Experimental Procedures—PET Image Acquisition and Analysis

PET scans with the radiotracer [18F]-2-deoxy-2-fluoro-D-glucose ([18F]-FDG) to measure regional cerebral glucose metabolism were acquired preoperatively and with the stimulators on after 1 and 12 months of continuous DBS. The PET scans were performed in 5 patients (numbers 2-6) on the CPS/Siemens high resolution research tomography (HRRT) scanner at the Centre for Addiction and Mental Health. [18F]-FDG was synthesized as described (Hamacher et al., 1986, Lemaire et al, 2002). During the radiotracer uptake period, subjects were maintained in a quiet, dimly lit room, with eyes open and ears unoccluded. 30 minutes after a 5 mCi±10% radiotracer injection, patients were positioned in the scanner and a twenty minute emission scan was obtained, followed by a transmission scan. The last ten minutes of the emission scan (40 minutes post-[18F]-FDG administration) were used for quantitative analysis.

Glucose metabolic rates were calculated (in ml/100 g/min) on a pixel by pixel basis by using a single venous blood sample (obtained 20 minutes after radiotracer injection) that is fit to a population curve (Takikawa et al., 1993). This quantification method has been validated against arterial blood sampling and is sensitive to disease and medication effects in AD (Takikawa et al., 1993, Smith et al., 2009). For the [18F]-FDG quantitative images, PET to PET registration was performed with statistical parametric mapping, version 5 (SPM5, Institute of Neurology, London) using the normalized mutual information algorithm. The images were spatially normalized into standard three-dimensional space relative to the anterior commissure using the MNI ICBM 152 stereotactic template within SPM5. Voxel-wise, statistical analyses were performed with SPM5. The glucose metabolism images were smoothed with an isotropic Gaussian kernel (FWHM 4 mm). The glucose metabolic rates were normalized by scaling to a common mean value across all scans, after establishing that the global means did not differ significantly across conditions (p>0.1). A between-subject comparison of baseline cerebral glucose metabolism in the AD patients and six demographically matched normal controls (age 68.5±9, gender 2 females/4 males) was performed using a two-sample t-test to evaluate the preoperative pattern of deficits in cerebral glucose metabolism in the AD group. A subject comparison of the baseline, one month and one year post-DBS conditions was performed using the flexible factorial option (paired t-test) in SPM5. For all analyses, the comparisons were considered significant at a t threshold greater than 3.51 (z>2.98, p<0.001; uncorrected for multiple independent comparisons) and was reported if the cluster size (KE) was greater than 50 voxels. Brain locations are reported as x, y, z coordinates in MNI space with approximate Brodmann areas (BA) identified by mathematical transformation of SPM5 coordinates into Tailarach space.

### Results—Patients

All 6 patients met diagnostic criteria for AD. As part of the inclusion criteria, all were on stable doses of acetyl cholinesterase inhibitors for a minimum of 6 months prior to study enrollment and throughout the 12 month study period. All patients scored 20 or higher on the screening MMSE test 3-4 months prior to surgery but in 2 patients, the MMSE score dropped to 15 and 19 in the preoperative assessment within 1 month of surgery. The subjects' demographics are shown in Table 1 immediately herebelow.

TABLE 1
Patient demographics, medication use and baseline MMSE scores
Results - Intraoperative findings with stimulation

| | | | | Baseline |
|---|---|---|---|---|
| Subject | Gender | Age | Medications | MMSE |
| 1 | F | 51 | Donepezil | 23 |
| 2 | F | 69 | Reminyl | 24 |
| 3 | M | 58 | Reminyl | 15 |
| 4 | M | 62 | Donepezil | 27 |
| 5 | M | 60 | Donepezil + | 19 |
| | | | Memantine | |
| 6 | M | 64 | Rivastigmine | 26 |
| MEAN | 2F, 4M | 60.7 | | 22.3 |

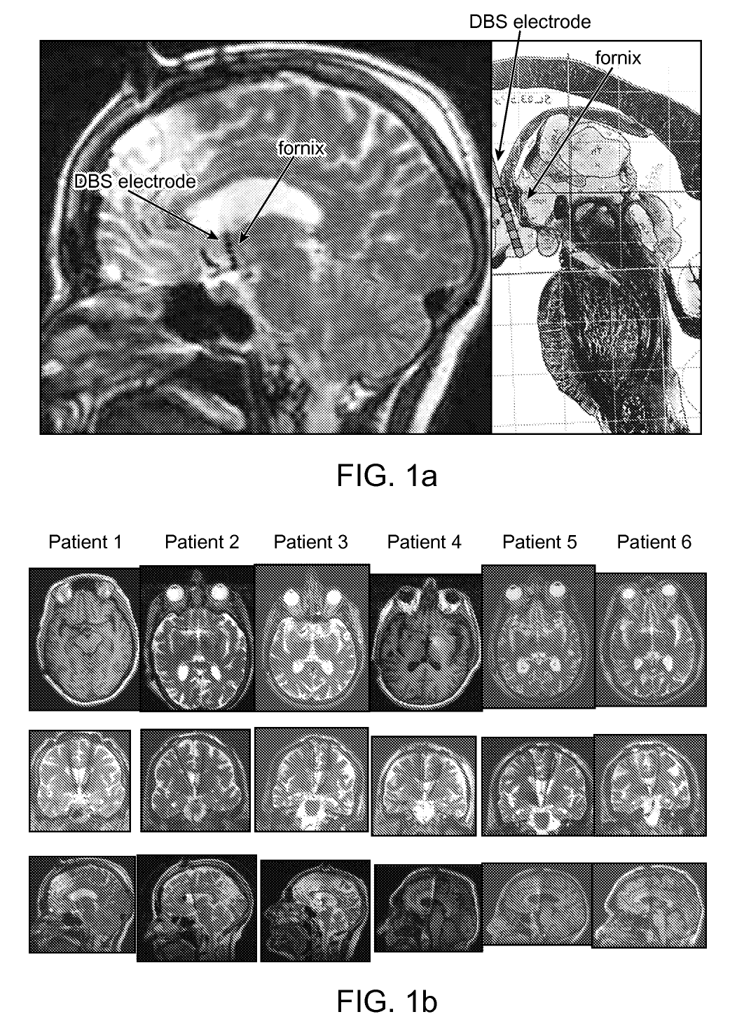

DBS electrodes were inserted first on the right and then the left, within the hypothalamus in contact with the anterior border of the vertical portion of the formix (FIG. 1a and 1b). Monopolar stimulation was applied after each electrode insertion at each of the 4 contacts at 130 Hz and 90 μsecond pulse widths increasing the voltage gradually, by 1.0 Volt every 30 seconds until an observable effect was reported or observed, or until the maximum intensity, 10 Volts was reached. Two of the six patients reported stimulation-induced “experiential” phenomena. Patient 2 reported having the sensation of being in her garden, tending to the plants on a sunny day with stimulation. In her case, this sensation outlasted the stimulation by several seconds. At certain contacts and settings, there was a pleasurable, warm sexual sensation that was clearly time-locked with the application of electrical stimulation. With stimulation, Patient 4 reported having the memory of being fishing on a boat on a wavy blue colored lake with his sons and catching a large green and white fish. On later questioning in both patients, these events were autobiographical, had actually occurred in the past and were accurately reported according to the patient's spouse. These sensations occurred at relatively high settings with thresholds of 5-6 Volts. In 5 subjects, increasing the current intensity to 7 to 10 volts produced a sensation of warmth accompanied by generalized flushing and increases in heart rate up to 120 per minute and increased in blood pressure by up to 20 mm Hg systolic. In 1 patient, patient 6, no acute stimulation effects were seen even at maximum currents.

### Results—Patient Outcomes

Applicant used the ADAS-cog/11 and changes in the MMSE as the primary measures to examine for the possible effects of stimulation on disease severity (Table 2). In general, surgery was well tolerated, with 3 patients showing a slight worsening (with increases in the ADAS-cog) and the other 3 showing a mild improvement with lowering in ADAS-cog scores after 1 month of stimulation compared to 1 month before surgery. After 6 months of stimulation, 4 of 6 patients showed improvement with lowering of 1.3 to 4.0 points in the ADAS-cog/11 scores. After 12 months of stimulation, 1 patient (number 4) continued to score 4.4 points lower on the ADAS-cog/11 than at baseline, 2 patients showed a 2 points increase, one patient a 5 point increase and 2 in other patients the scores increased by more than 5 points. The rate of change in the ADAS-cog scores in AD patients is variable, non-linear and controversial with historical figures suggesting an increase in the range of 3-10 points per year and a mean of 6-7 point increase per year (Mayeux and Sano, 1999). Overall, there was a mean increase of 4.2 points in the ADAS-cog/11 in the 6 DBS patients over 12 months. The expected change after 12 months of disease progression was calculated according to a regression formula based on a metaanalysis of over 50 studies involving more than 19,000 (Ito et al., 2010). Two of the patients experienced a less than expected increase in score, 1 more than expected, and in 3 patients the ADAS-cog/11 scores were within 2 points of the expected change after 12 months.

TABLE 2

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −52 | −50 | 13 | 3.49 | Superior | | | | |
| | | | | Temporal | | | | |
| | | | | Gyrus (BA | | | | |
| | | | | 22) | | | | |
| | | | | Middle | 51 | −69 | 11 | 5.03 |
| | | | | Temporal | | | | |
| | | | | Gyrus (BA | | | | |
| | | | | 39) | | | | |
| | | | | Middle | 60 | −41 | −15 | 4.48 |
| | | | | Temporal | | | | |
| | | | | Gyrus (BA | | | | |
| | | | | 21) | | | | |
| | | | | Precuneus | 6 | −67 | 33 | 3.89 |
| | | | | (BA 7) | | | | |
| −13 | −68 | 25 | 3.38 | Posterior | | | | |
| | | | | Cingulate | | | | |
| | | | | (BA 31) | | | | |
| −54 | −52 | −18 | 3.94 | Fusiform | 49 | −66 | 1 | 4.73 |
| | | | | Gyrus (BA | | | | |
| | | | | 37) | | | | |
| | | | | Angular | 47 | −69 | 33 | 4.16 |
| | | | | Gyrus (BA | | | | |
| | | | | 39) | | | | |
| −56 | −45 | 40 | 4.52 | Inferior | 59 | −44 | 41 | 4.82 |
| | | | | Parietal | | | | |
| | | | | Lobule (BA | | | | |
| | | | | 40) | | | | |
| −27 | −71 | 34 | 5.04 | Cuneus (BA | | | | |
| | | | | 19) | | | | |

MMSE and ADAS-cog/11 scores for 6 patients at baseline (Preop) and after 1, 6 or 12 months of fornix/hypothalamic deep brain stimulation. The 12-month predicted scores are based on a regression formula from a meta-analysis by Ito et al., 2010. Based on this measure, 2 patients (1 and 4) had less than expected progression, 3 patients scored within 2 points of expected (2, 5 and 6) and 1 patient (3) deteriorated more than expected.

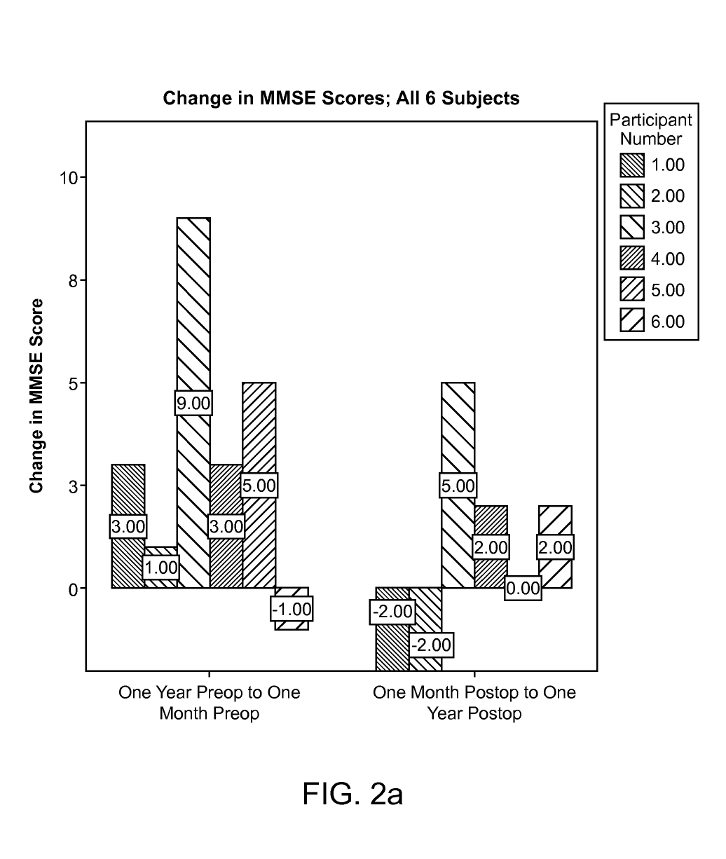

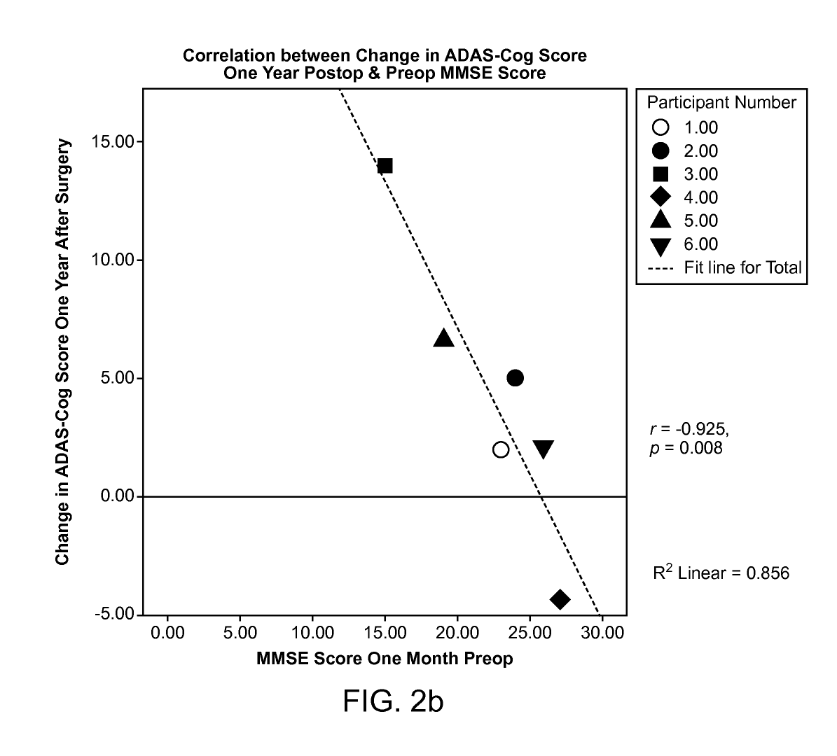

To support these observations, applicant also assessed changes in MMSE scores over time. The change in MMSE score from baseline to 12 months ranged from an improvement of 2 points to a decline of 8. The expected rate of decline in this population is also variable and difficult to ascertain but has been estimated at approximately 10% of total score of 30, or 3 points/year (Mayeux and Sano, 1999). In comparing the rate of decline in the 11 months preceding surgery to the 11 months after surgery a decrease was seen in the rate of decline from a mean rate of 2.8 to 0.8 points across the 6 patients (FIG. 2a). With the caveat that there is no appropriate contemporary control group with which to compare, together, the ADAS-cog and MMSE data suggest improvement in the rate of decline with DBS in certain patients.

Applicant examined whether there was a relationship between disease severity and likelihood of benefit with DBS, and found a strong correlation (r=−0.925 and p=0.008) between preoperative cognitive function as assessed with MMSE or ADAS-cog and the propensity for response to DBS with the least affected patients having less decline in ADAS-cog scores after 12 months of stimulation (FIG. 2b). It should be noted however that the rate of change in ADAS-cog scores in AD is non-linear with a tendency for the less severe and the most advanced patients to show lesser decline (Stern et al, 1994). On the other hand, patient 3 who was the most severely affected at baseline and had shown rapid progression with a fall in the MMSE of 9 points in the year prior to DBS, showed the largest decline in the MMSE in the year following surgery.

Because the formix is within the circuit mediating memory, applicant considered the possibility that DBS at this site would have a preferential effect on memory function. The ADAS-cog scale measures memory, language and praxis domains. Applicant sought to determine whether there was a selective effect in any of these domains with stimulation. In the 3 patients showing improvement or the least increase in the ADAS-cog scores after 12 months of DBS (Nos. 1, 4 and 6), but not in the other subjects, the amelioration in the ADAS-cog scores with DBS was driven by improvement in the recall and recognition components of the ADAS-cog (data not shown). For patient 4, who had the highest baseline and the greatest improvement in score with DBS, the benefits were exclusively in these components of the ADAS-cog. These observations suggest the possibility that DBS may be driving the function of this memory circuit. The differential response across subjects suggests a possible relation between disease severity and the functional integrity of the formix-hippocampus circuit and the propensity for benefit with DBS.

Applicant also assessed the impact of formix DBS on global function and quality of life measures. The changes in the cognitive measures were accompanied by 2 to 5 point improvements in the AD specific QOL scale at 12 months in 4 of 6 patients (Nos. 1-4). While suggestive of a benefit, the significance of the QOL measures is not clear due to their variable relation to cognitive function and the lack of contemporaneous control patients. Consistent with the quality of life literature in dementia (Vogel et al., 2006), the patients reported better overall outcomes than their spouses. On the Clinician Interview Based Impression of Change (CIBIC) scale, a global measure of outcome, 4 subjects reported no change after 12 months, 1 reported minimal improvement and 1 reported minimal worsening. In comparison, 2 patients were said to show no changes and 4 to be minimally worse as assessed by the informant and a treating neurologist at 1 year.

Results—sLORETA

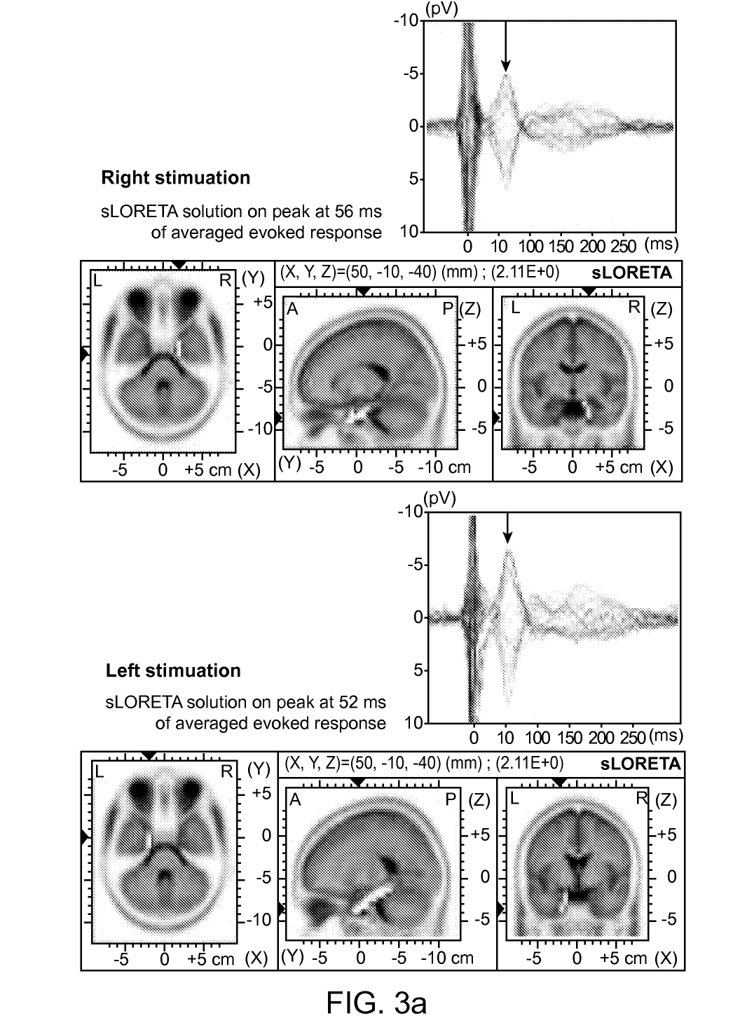

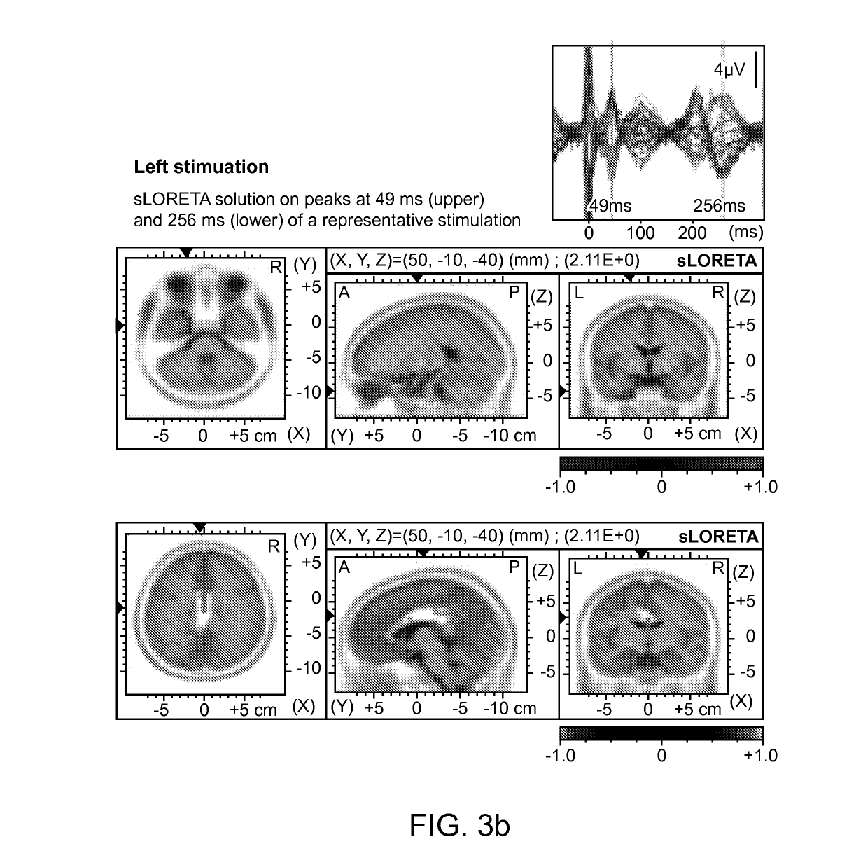

Applicant hypothesized that stimulation of the formix/hypothalamus would drive activity in downstream projection structures, and used sLORETA to identify and map which brain areas were affected by electrical stimulation. Stimulation of the formix/hypothalamus through the implanted DBS electrodes led to short latency specific and localized changes in the activity of ipsilateral mesial temporal lobe structures. Across the 6 patients, the peak of the first obvious evoked response after stimulation had a latency of 38 to 52 ms and was localized to hippocampus and parahippocampal gyms (FIG. 3a). The evoked responses and their sources were unequivocal and consistent with all patients showing a similar pattern. The evoked response was absent on the right side in one patient (patient 3) whose right electrode was situated in the ventricle adjacent to the hypothalamus and formix (FIG. 1b). At longer latencies after stimulation (102 to 256 ms), significant activation of the cingulate gyrus and precuneus area of the parietal lobe were seen (FIG. 3b). The changes were almost exclusively ipsilateral to the side of stimulation. These findings are consistent with the direct and trans-synaptic sequential activation of downstream targets related to the known connectivity of the formix and hippocampus and shows that DBS drives activity in this important memory circuit and the closely synaptically connected brain's downstream default mode network.

### Results—PET

PET measures of cerebral glucose metabolism were used to characterize the activity of brain networks preoperatively and to provide topographic and quantitative measures of the effects of DBS. Resting state scans were performed before surgery and after 1 and 12 months of continuous formix/hypothalamic DBS. DBS remained on during the scans. The results of the voxel-wise analyses of the comparison between AD patients and controls and the comparison in the AD patients between baseline to 1 month DBS, baseline to 12 months DBS and 12 month DBS to 1 month DBS conditions is shown in Tables 3A-3G herebelow.

TABLE 3A

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −52 | −50 | 13 | 3.49 | Superior | | | | |
| | | | | Temporal | | | | |
| | | | | Gyrus (BA | | | | |
| | | | | 22) | | | | |
| | | | | Middle | 51 | −69 | 11 | 5.03 |
| | | | | Temporal | | | | |
| | | | | Gyrus (BA | | | | |
| | | | | 39) | | | | |
| | | | | Middle | 60 | −41 | −15 | 4.48 |
| | | | | Temporal | | | | |
| | | | | Gyrus (BA | | | | |
| | | | | 21) | | | | |
| | | | | Precuneus | 6 | −67 | 33 | 3.89 |
| | | | | (BA 7) | | | | |
| −13 | −68 | 25 | 3.38 | Posterior | | | | |
| | | | | Cingulate | | | | |
| | | | | (BA 31) | | | | |
| −54 | −52 | −18 | 3.94 | Fusiform | 49 | −66 | 1 | 4.73 |
| | | | | Gyrus (BA | | | | |
| | | | | 37) | | | | |
| | | | | Angular | 47 | −69 | 33 | 4.16 |
| | | | | Gyrus (BA | | | | |
| | | | | 39) | | | | |
| −56 | −45 | 40 | 4.52 | Inferior | 59 | −44 | 41 | 4.82 |
| | | | | Parietal | | | | |
| | | | | Lobule (BA | | | | |
| | | | | 40) | | | | |
| −27 | −71 | 34 | 5.04 | Cuneus (BA | | | | |
| | | | | 19) | | | | |

TABLE 3B

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −36 | −18 | 49 | 3.79 | Precentral | | | | |
| | | | | Gyrus (BA 4) | | | | |
| −38 | 0 | 14 | 3.67 | Insula | | | | |
| −42 | −30 | 7 | 3.41 | Superior | | | | |
| | | | | Temporal | | | | |
| | | | | Gyrus (BA 41) | | | | |
| | | | | Middle | 61 | −25 | −7 | 4.46 |
| | | | | Temporal | | | | |
| | | | | Gyrus (BA 21) | | | | |
| −52 | −29 | 1 | 3.74 | Middle | 63 | −33 | 4 | 4.02 |
| | | | | Temporal | | | | |
| | | | | Gyrus (BA 22) | | | | |
| | | | | Inferior | 55 | −57 | −14 | 3.56 |
| | | | | Temporal | | | | |
| | | | | Gyrus (BA 20) | | | | |
| | | | | Inferior | 52 | −67 | 2 | 3.74 |
| | | | | Temporal | | | | |
| | | | | Gyrus (BA 37) | | | | |
| −35 | −49 | −14 | 3.94 | Fusiform Gyrus | 47 | −65 | −12 | 3.04 |
| −28 | −36 | 60 | 3.78 | Post-central | | | | |
| | | | | Gyrus (BA 2) | | | | |
| −44 | −18 | 50 | 3.71 | Post-central | | | | |
| | | | | Gyrus (BA 3) | | | | |
| | | | | Superior | 25 | −42 | 59 | 4.55 |
| | | | | Parietal Lobule | | | | |
| | | | | (BA 5) | | | | |
| −20 | −68 | 56 | 4.36 | Superior | 18 | −63 | 59 | 3.68 |
| | | | | Parietal Lobule | | | | |
| | | | | (BA 7) | | | | |
| 10 | −50 | 48 | 3.39 | Precuneus (BA | | | | |
| | | | | 7) | | | | |
| −18 | −72 | 20 | 4.29 | Posterior | | | | |
| | | | | Cingulate (BA | | | | |
| | | | | 31) | | | | |
| −21 | −40 | 58 | 3.8 | Inferior Parietal | | | | |
| | | | | Lobule (BA 40) | | | | |
| −9 | −96 | −13 | 3.44 | Lingual Gyrus | | | | |
| | | | | (BA 17) | | | | |
| −31 | −94 | −5 | 4.12 | Lingual Gyrus | 2 | −89 | −2 | 4.1 |
| | | | | (BA 18) | | | | |
| −37 | −80 | −14 | 3.41 | Cuneus (BA | 9 | −91 | 25 | 3.86 |
| | | | | 19) | | | | |
| | | | | Medulla | 7 | −39 | −36 | 4.05 |
| −18 | −51 | −25 | 3.7 | Cerebellum | 20 | −52 | −26 | 3.35 |
| | | | | (Dentate) | | | | |
| −27 | −53 | −18 | 4.32 | Cerebellum | 20 | −45 | −17 | 3.73 |
| | | | | (Culmen) | | | | |
| −6 | −53 | −20 | 3.33 | Cerebellum | 6 | −55 | −22 | 3.97 |
| | | | | (Fastigium) | | | | |
| | | | | Cerebellum | 15 | −58 | −18 | 4.21 |
| | | | | (Declive) | | | | |

TABLE 3C

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −10 | 6 | 35 | 4 | Anterior | 3 | −17 | 40 | 4.15 |
| | | | | Cingulate | | | | |
| | | | | Gyrus (BA 24) | | | | |
| −5 | 40 | 13 | 3.36 | Anterior | 4 | 37 | −6 | 4.03 |
| | | | | Cingulate (BA | | | | |
| | | | | 32) | | | | |
| −28 | 48 | −4 | 4.03 | Medial Frontal | 4 | 50 | 12 | 3.51 |
| | | | | Gyrus (BA 10) | | | | |
| −25 | 42 | 38 | 4.23 | Middle Frontal | 27 | 30 | 39 | 3.59 |
| | | | | Gyrus (BA 8) | | | | |
| −55 | 9 | 9 | 3.33 | Precentral | 49 | −2 | 48 | 3.64 |
| | | | | Gyrus (BA 6) | | | | |
| −15 | 11 | 10 | 3.26 | Caudate | | | | |
| −18 | −1 | 0 | 3.66 | Lateral Globus | | | | |
| | | | | Pallidus | | | | |
| −7 | −12 | 7 | 3.55 | Thalamus | | | | |
| | | | | (Medial Dorsal | | | | |
| | | | | Nucleus) | | | | |

TABLE 3D

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −18 | −5 | 58 | 4.29 | Middle | | | | |
| | | | | Frontal | | | | |
| | | | | Gyrus (BA | | | | |
| | | | | 6) | | | | |
| −4 | −36 | 50 | 3.22 | Paracentral | 4 | −34 | 50 | 3.36 |
| | | | | Lobule (BA | | | | |
| | | | | 5) | | | | |
| −48 | −9 | 51 | 3.92 | Pre-central | 36 | −13 | 55 | 3.78 |
| | | | | Gyrus (BA | | | | |
| | | | | 4) | | | | |
| | | | | Superior | 59 | −26 | 9 | 3.63 |
| | | | | Temporal | | | | |
| | | | | Gyrus (BA | | | | |
| | | | | 41) | | | | |
| −61 | −43 | 12 | 3.85 | Superior | | | | |
| | | | | Temporal | | | | |
| | | | | Gyrus (BA | | | | |
| | | | | 22) | | | | |
| −60 | −35 | 5 | 3.95 | Middle | | | | |
| | | | | Temporal | | | | |
| | | | | Gyrus (BA | | | | |
| | | | | 22) | | | | |
| −6 | −67 | 14 | 3.42 | Posterior | | | | |
| | | | | Cingulate | | | | |
| | | | | (BA 31) | | | | |
| −55 | −26 | 41 | 4.12 | Post- | 54 | −26 | 44 | 3.94 |
| | | | | central | | | | |
| | | | | Gyrus (BA | | | | |
| | | | | 2) | | | | |
| −20 | −65 | 51 | 3.32 | Precuneus | 16 | −51 | 57 | 3.75 |
| | | | | (BA 07) | | | | |
| −32 | −40 | 55 | 4.07 | Inferior | 47 | −33 | 43 | 3.42 |
| | | | | Parietal | | | | |
| | | | | Lobule (BA | | | | |
| | | | | 40) | | | | |
| −6 | −70 | 5 | 4.16 | Lingual | 5 | −88 | 19 | 4.37 |
| | | | | Gyrus (BA | | | | |
| | | | | 18) | | | | |
| | | | | Lingual | 16 | −60 | −2 | 3.41 |
| | | | | Gyrus (BA | | | | |
| | | | | 19) | | | | |
| −32 | −20 | 11 | 4.02 | Claustrum | 34 | −18 | 7 | 3.44 |
| −28 | 3 | 10 | 4.02 | Putamen | | | | |
| −12 | −18 | 1 | 3.68 | Thalamus | | | | |
| | | | | (Mammillary | | | | |
| | | | | Body) | | | | |
| | | | | Thalamus | 17 | −23 | 12 | 3.93 |
| | | | | (Pulvinar) | | | | |
| | | | | Cerebellum | 16 | −60 | −21 | 4.40 |
| | | | | (Dentate) | | | | |
| −20 | −53 | −37 | 4.3 | Cerebellar | 9 | −52 | −34 | 3.98 |
| | | | | Tonsil | | | | |
| −9 | −75 | −11 | 4.31 | Cerebellum | 16 | −83 | −17 | 4.15 |
| | | | | (Declive) | | | | |

TABLE 3E

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −1 | −22 | 33 | 3.93 | Anterior | 3 | −33 | 29 | 4.14 |
| | | | | Cingulate | | | | |
| | | | | Gyrus (BA 23) | | | | |
| −7 | −12 | 38 | 4.0 | Anterior | | | | |
| | | | | Cingulate | | | | |
| | | | | Gyrus (BA 24) | | | | |
| | | | | Anterior | 6 | 37 | 26 | 3.94 |
| | | | | Cingulate (BA | | | | |
| | | | | 32) | | | | |
| | | | | Medial Frontal | 6 | 49 | 10 | 3.88 |
| | | | | Gyrus (BA 10) | | | | |
| −17 | 13 | 61 | 4.7 | Middle Frontal | 4 | 28 | 36 | 3.57 |
| | | | | Gyrus (BA 6) | | | | |
| | | | | Inferior Frontal | 42 | 20 | 12 | 3.65 |
| | | | | Gyrus (BA 45) | | | | |

TABLE 3F

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −7 | 26 | 33 | 3.6 | Anterior | | | | |
| | | | | Cingulate | | | | |
| | | | | Gyrus (BA 32) | | | | |
| −40 | −1 | 46 | 4.19 | Medial Frontal | 7 | 16 | 45 | 4.42 |
| | | | | Gyrus (BA 6) | | | | |
| −42 | 12 | 34 | 4.23 | Middle Frontal | 46 | 24 | 33 | 3.79 |
| | | | | Gyrus (BA 9) | | | | |
| −52 | −14 | 41 | 3.76 | Pre-central | 46 | −14 | 46 | 3.28 |
| | | | | Gyrus (BA 4) | | | | |
| −51 | −4 | 38 | 3.81 | Pre-central | | | | |
| | | | | Gyrus (BA 6) | | | | |
| −10 | 23 | −12 | 4.37 | Subcallosal | | | | |
| | | | | Gyrus (BA 11) | | | | |
| −57 | −26 | 28 | 3.94 | Inferior Parietal | 40 | −30 | 41 | 4.09 |
| | | | | Lobule (BA 40) | | | | |
| −51 | −21 | 46 | 4.02 | Post-central | 49 | −28 | 41 | 3.57 |
| | | | | Gyrus (BA 2) | | | | |
| −19 | −4 | −2 | 3.4 | Globus Pallidus | | | | |
| 16 | −21 | 3 | 3.72 | Thalamus | | | | |
| | | | | (Ventral | | | | |
| | | | | Posterior | | | | |
| | | | | Medial | | | | |
| | | | | Nucleus) | | | | |
| −7 | −54 | −24 | 3.62 | Cerebellum | | | | |
| | | | | (Dentate) | | | | |
| −22 | −50 | −37 | 3.81 | Cerebellum | | | | |
| | | | | (Posterior | | | | |
| | | | | Lobe) | | | | |
| −14 | −62 | −17 | 3.57 | Cerebellum | | | | |
| | | | | (Declive) | | | | |

TABLE 3G

| Left Hemisphere | | | | | Right Hemisphere | | | |
|---|---|---|---|---|---|---|---|---|
| x (mm) | y (mm) | z (mm) | Z score | Structure | x (mm) | y (mm) | z (mm) | Z score |
| −2 | 55 | −4 | 3.74 | Medial Frontal | | | | |
| | | | | Gyrus (BA 10) | | | | |
| | | | | Cingulate | 6 | −27 | 33 | 3.4 |
| | | | | Gyrus (BA 23) | | | | |
| | | | | Subcallosal | 9 | 22 | −13 | 3.94 |
| | | | | Gyrus (BA 25) | | | | |
| | | | | Superior | 53 | −50 | 12 | 4.72 |
| | | | | Temporal | | | | |
| | | | | Gyrus (BA 22) | | | | |
| | | | | Middle | 56 | −9 | −6 | 3.52 |
| | | | | Temporal | | | | |
| | | | | Gyrus (BA 21) | | | | |
| | | | | Fusiform Gyrus | 50 | −57 | −16 | 3.53 |
| | | | | (BA 37) | | | | |
| | | | | Inferior | 52 | −66 | 2 | 4.26 |
| | | | | Temporal | | | | |
| | | | | Gyrus (BA 37) | | | | |
| | | | | Parahippocampal | 31 | −33 | −14 | 3.35 |
| | | | | Gyrus (BA | | | | |
| | | | | 36) | | | | |
| −11 | −52 | −8 | 3.8 | Cerebellum | 16 | −49 | −4 | 3.78 |
| | | | | (Culmen) | | | | |
| | | | | Cerebellum | 42 | −73 | −17 | 3.99 |
| | | | | (Declive) | | | | |

TABLES 3A-3G: Tables comparing cerebral glucose utilization in 5 AD patients (2-6)-versus age-matched healthy control subjects. The table tracks changes in glucose utilization from baseline to after 1 month of DBS and from baseline and after 1 month of DBS to 12 months of DBS. PET scans were obtained after 1 and 12 months of continuous DBS and were acquired with the stimulation on.

Consistent with previous studies, the AD patients showed a reduction in glucose metabolism particularly in the temporal and parietal regions compared to healthy controls (see Tables 3A-3G hereabove). Significant metabolic decreases in AD patients compared to controls was observed in left superior temporal (BA 22), right middle temporal (BA 21, BA 39), right precuneus (BA 7) and right angular gyrus (BA 39), left posterior cingulate (BA 31) and cuneus (BA 19) and bilateral fusiform gyrus (BA 37) and inferior parietal lobule (BA 40).

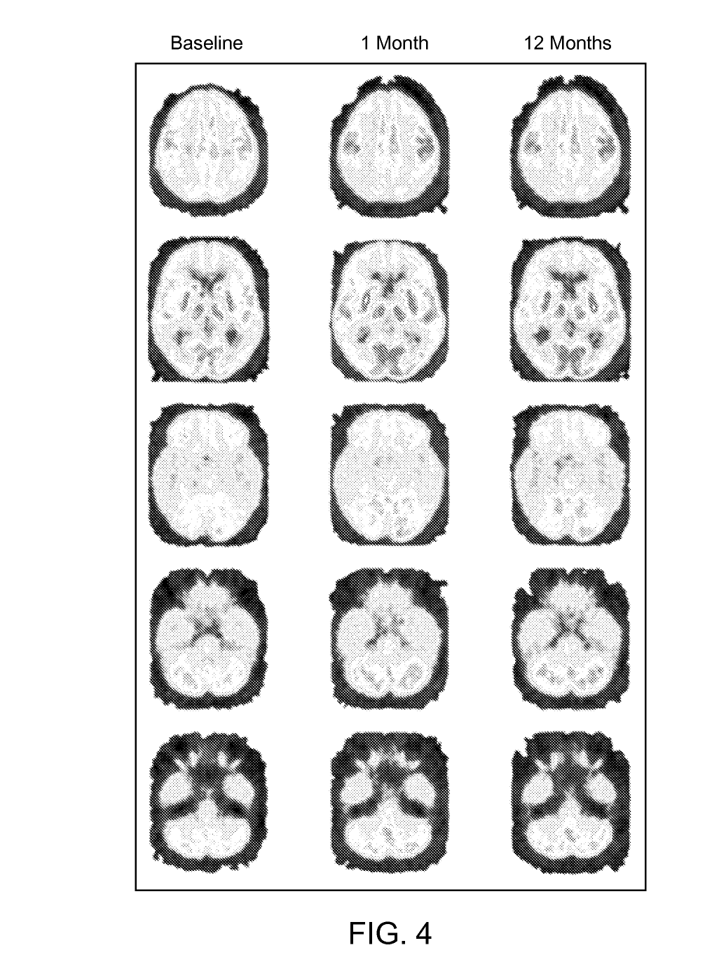

DBS was accompanied by widespread changes in metabolic activity in cortical and subcortical areas (FIG. 4 and Tables 3A-3G). The comparison of one month DBS to baseline showed increased metabolism in left pre-central gyrus [BA4] and left insula, temporal (left superior [BA 41], right [BA21] and bilateral [BA 22] middle, right [BA 20] and left [BA 37] inferior and left fusiform) gyri, parietal (right [BA 5] and bilateral [BA7] superior parietal lobule, left precuneus [BA7], left posterior cingulate [BA 31], left inferior parietal lobule [BA 40]) gyri, occipital (left [BA 17] and bilateral lingual [BA 18] and bilateral cuneus [BA 19]) gyri, left medulla and cerebellum (bilateral dentate, culmen and fastigum and right declive). Decreased metabolism was observed in anterior cortical areas including the anterior cingulate [BA 24, 32], bilateral medial and middle frontal [BA 8, 10] and bilateral pre-central [BA 6] gyri and sub-cortical areas including the left caudate, left lateral globus pallidus and left thalamus (medial dorsal nuclei).

The pattern of regional glucose metabolism after 1 year of constant DBS was examined to determine whether the changes seen after 1 month of stimulation were sustained and to ascertain the effects of chronic stimulation in the context of the known progressive nature of the degeneration in AD. In contrast to the known transient effect seen with the ongoing daily use of medications, the comparison of one year DBS to baseline showed that the increased metabolism seen after 1 month of stimulation persisted in many of the most affected brain areas. Specifically, increased metabolism was observed in frontal (left middle frontal [BA 6], bilateral paracentral lobule [BA 5] and bilateral pre-central [BA4]) gyri, temporal (right superior temporal [BA 41], left superior and middle temporal [BA 22]) gyri, parietal (left posterior cingulate [BA 31], bilateral precuneus [BA 7], bilateral post-central [BA 2] and bilateral inferior parietal lobule [BA 40]) gyri. Increased metabolism was also observed in the cerebellum (bilateral tonsil and declive) and subcortical areas including the bilateral claustrum, left putamen and thalamic nuclei (left mammilary bodies and right pulvinar). Decreased metabolism was observed at 12 months in the anterior cingulate (bilateral BA 23, left BA 24, right BA 32) gyri, bilateral [BA 6] and left [BA10] and right inferior frontal gyri [BA 45].

The comparison of one year to one month DBS showed increased metabolism in left anterior cingulate gyrus [BA 32], middle frontal gyrus (bilateral BA 6 and 9 and left BA 10), pre-central gyms (bilateral BA 4 and left BA 6), left sub-callosal gyms (BA 11), bilateral inferior parietal lobule [BA 40] and post-central gyms [BA 2], left globus pallidus, left thalamus and left cerebellum (dentate, posterior lobe, declive). Decreased metabolism was observed in right anterior [BA 23] and subcallosal [BA 25] cingulate gyms, left medial frontal gyrus [BA 10] and right superior, middle and inferior temporal gyrus, right fusiform, right parahippocampal gyrus and cerebellum (bilateral culmen and right declive). While many temporal and parietal regions showed persistent metabolic increases after one year DBS relative to baseline, the comparison of one year to one month revealed increased metabolism in anterior cortical and subcortical regions not affected at baseline.

The brain areas that demonstrated the greatest increases in metabolism with DBS are among those known to have large accumulations of amyloid deposits, the greatest impairment in glucose utilization and the greatest physiologic dysfunction in AD patients (Buckner et al., 2005). DBS also produced long lasting increases in glucose utilization in the posterior cingulated lobe, parietal lobe and, precuneus, which are important components of the brain default mode network that are most affected early in the course of AD. The results shown here indicate that formix/hypothalamic DBS produces striking and sustained changes in cognitive and limbic brain areas and modulates the activity of the default network and they provide a possible biological basis for the observed changes in certain AD patients.

### Results—Adverse Effects

Surgery was well tolerated. Patients were discharged 1-3 days after surgery. Stimulation related adverse effects were autonomic and cardiovascular in nature and occurred at high stimulation settings. In dose finding experiments, at the upper levels of stimulation at 7 to 10 Volts, 5 of 6 patients experienced a sensation of warmth, flushing and sweating. In 3 patients there were increases in heart rate and blood pressure seen at stimulation over 7 volts. Chronic stimulation settings were chosen at levels approximately 50% of the voltage threshold for adverse effects. After the initial surgery, no patient required hospitalization during the 12 months of the study.

### Discussion—Principal Findings

The application of DBS in the hypothalamus and formix of these 6 patients with mild Alzheimer's disease was safe and produced biological effects. Stimulation was associated with potential benefits in that it drove physiologic activity and produced large and sustained changes in glucose metabolism in brain regions that were dysfunctional. With the caveat that this pilot trial is open label and uncontrolled, there is a suggestion that the rate of cognitive decline after surgery is diminished after DBS in these patients. There is also the suggestion that less severely affected patients are perhaps more likely to benefit, due to having more of the integrity of the circuitry preserved. Indeed the early evidence suggests a clear relation with less severely affected patients less likely to decline after DBS (FIG. 2b).

### Discussion—Anatomical and Functional Specificity of Stimulation

The brain regions that showed the most prominent changes in their electrophysiologic activity with stimulation were specific and synaptically connected within the circuit of Papez and the downstream default mode network. The sLORETA analysis showed that stimulation produced strong early ipsilateral activation in the hippocampus and mesial temporal lobe, structures intimately involved in memory function. This finding made us consider the possibility that formix/hypothalamic stimulation could have a preferential affect on the recall and recollection. Indeed the improvement in ADAS-cog scores were driven predominantly by improvements in memory related measures of the 11 component ADAS-cog scale (ADAS-cog/11), in the patients (1 and 4) showing the improvement or less than expected decline with DBS (Table 2). In addition, it was found that patients who were less severely affected were more likely to be “responders” to formix/hypothalamic DBS and show improvement or less than expected decline in cognitive function (FIG. 2 a,b). These observations are consistent with the notions that better preserved circuit integrity as may be expected in milder patients, may be a predictor of response to DBS and that stimulation at the formix/hypothalamic site may show preferential effects on memory function versus some of the other elements of cognitive function that are impaired in AD.

| | | | | | 12 | 12 |
|---|---|---|---|---|---|---|
| | | | 1 Month | 6 Months | Months | Months |
| | | Preop | Postop | Postop | Postop | Predicted |
| | Preop | ADAS- | ADAS- | ADAS- | ADAS- | ADAS- |
| Patient | MMSE | Cog | Cog | Cog | Cog | Cog |
| 1 | 23 | 18.7 | 16.0 | 16.3 | 20.7 | 23.2 |
| 2 | 24 | 18.3 | 24.3 | 17.7 | 23.3 | 22.7 |
| 3 | 15 | 28.7 | 32.0 | 38.0 | 42.7 | 34.7 |
| 4 | 27 | 11.7 | 11.3 | 7.7 | 7.3 | 15.0 |
| 5 | 19 | 24.0 | 27.7 | 27.0 | 30.7 | 29.3 |
| 6 | 26 | 13.3 | 12.7 | 12.0 | 15.3 | 16.9 |

### Discussion—Effects on the Default Mode Network

The sLORETTA studies showed stimulation of the formix/hypothalamus produced clear and specific activation of the posterior cingulate and medial parietal lobe with a mean latency of 250 ms, approximately 200 ms after activation of the mesial temporal structures (FIGS. 3a, 3b). Thus, both the sLORETA findings (FIGS. 3a, 3b) supported by the PET metabolic studies (FIG. 4 and Tables 3A-3G) show that DBS has effects on brain areas which closely overlap with brain default mode network. This network, components of which include the medial temporal lobe, part of the medial prefrontal cortex and the posterior cingulate cortex along with the adjacent precuneus and the medial, lateral and inferior parietal cortex was identified on the basis of coherent low frequency (less than 0.1 Hz) neuronal oscillations and is so named because it preferentially activates when individuals focus on internal tasks such as daydreaming, envisioning the future, retrieving autobiographical memories, and gauging others' perspectives (Raichle 2001; Buckner 2009). Regions within the default network show structural and functional connectivity that converges on the posterior cingulate extending into the precuneus which is strongly interconnected with the hippocampal formation (Grecius et al., 2004, Buckner 2009). The observation in AD patients of reduced resting state metabolism and of impaired functional deactivation in the default network during memory tasks in subjects of advanced age and particularly with brain amyloid accumulation (Greicius et al., 2004; Sperling et al., 2010), provide additional support to the hypothesis this circuit plays a critical role in modulating memory functions. The consequences of modulating neural activity of this network are not known but the ability to reach and influence the activity of this network with DBS introduces an interesting, adjustable and reversible means of changing the activity of this network and potentially ascertaining its function(s).

### Discussion—PET Changes Compared to Other Interventions in AD

DBS reversed the reduced cortical glucose utilization in the temporal and parietal regions in the AD patients (Tables 3A-3G). The increased cerebral glucose metabolism relative to baseline was observed at one month after DBS treatment and persisted to one year. The increases in metabolism were observed in temporal and parietal cortical areas that are the earliest and most severely affected in the course of AD (Minoshima, et al., 1997, Reiman et al., 1996) as well as in related downstream connections, the precuneus and the posterior cingulate regions, important components of the default network. The increases in cerebral metabolism in temporal cortical regions are greater at one month compared to one year post DBS. However, at one year, metabolism was still increased relative to baseline in an extensive network of temporal and parietal regions. The areas of increase included not only the cortical heteromodal association areas that are most affected in AD, but also included the primary sensory and motor cortices, striatum, thalamus and cerebellar regions that show relatively lower levels of AD pathology and are relatively spared in previous cerebral glucose metabolism studies in AD (Arnold et al., 1991, Smith et al., 1992). Increased metabolism was observed in pre and postcentral gyms, occipital cortex and cerebellum, all regions that are relatively less affected in AD (including the subject enrolled in this study), even in the advanced stages of the illness. Thus, DBS increased metabolism in the regions affected in AD, but in addition, DBS also had effects along more widely distributed cortical regions that are relatively spared in AD, presumably related to subcortical-cortical or cortical-cortical trans-synaptic effects of stimulation. DBS was also associated with decreased metabolism in sub regions of the anterior cingulate and the medial frontal cortices. Dysfunction here may manifest as impairments in executive function tasks.

The cerebral metabolic effects of DBS should be interpreted relative to the long term effects of pharmacotherapy (acetylcholinesterase inhibitors) on cerebral metabolism, as well as the longitudinal decline in cerebral metabolism over one year. While the short term effects of cholinesterase inhibitors have been studied, there are few reports of longer treatment duration. Several studies have shown short-term metabolic increases by cholinesterase inhibitors, including donepezil, galantamine and phenserine (Tune, et al., 2003, Smith et al., 2009, Kadir et al., 2008). The increases are observed in frontal and parieto-temporal cortical areas that are most affected in AD. The increases in metabolism are observed over a relatively short interval (3 months) and metabolism returns to pre-treatment levels after longer term treatment (6 months; Tune, et al., 2003, Kadir et al, 2008). One year of rivastigmine treatment was associated with a significant increase in cerebral glucose metabolism in right frontal association cortex, right putamen and globus pallidus and bilateral cingulate gyrus (BA 24, 32) (Stefanova et al., 2006). The other cortical regions showed nonsignificant increases in metabolism relative to baseline in contrast to the untreated AD group that showed significant decreases in metabolism in cortical association areas. Long term rivastigmine treatment prevented the decline in cerebral glucose metabolism over the course of one year.

In contrast to the effects of cholinesterase inhibitors, both one month and one year of DBS was associated with an increase in cerebral glucose metabolism relative to baseline in a network of brain regions that was much more extensive than that observed with cholinesterase inhibitor treatment. Over the course of one to two year follow-up, AD patients consistently show progressive metabolic decreases in cortical association areas, with relative sparing of primary sensory (visual, somatosensory) and motor cortical areas, basal ganglia, thalamus and cerebellum. (Stefanova et al. 2006; Smith et al, 1992; Alexander, et al., 2002). The progressive metabolic decline observed over the one year course of AD further underscores the significance of the extensive metabolic increases associated with DBS.

### Discussion—Choosing Stimulation Parameters and Mechanism of Action

Stimulation parameters were chosen empirically and there may be opportunities for optimization. The starting point for stimulation was 3.5 V, 130 Hz and pulse width of 90 microseconds—settings that are similar to those used in DBS for PD. An increase in the intensity of stimulation was made until adverse effects were encountered—usually at 5-8 volts and reduced stimulation by 50%. Applicant used several observations in choosing the parameters for chronic stimulation. First, as a dose-response relationship was detected between stimulation and the magnitude of change in the EEG as seen by the sLORETA analysis, applicant wanted to use relatively high settings. Second, applicant wanted stimulation to be free of any perceived sensations or adverse effects. Applicant opted for continuous uninterrupted stimulation as is used in PD and dystonia DBS. Circuitry changes may be a consequence of prolonged stimulation or ongoing stimulation may be necessary after a prolonged period of stimulation. In a preliminary experiment, stimulation was halted for a period of 1 month in a double blinded fashion in 5 patients after 12 months of stimulation and a major decline in ADAS-cog measures was not observed. While there are many explanations, the possibility of a long lasting washout effect or of enduring changes in circuit properties as a consequence of the stimulation needs to be considered.

While there is degeneration in the Papez circuit in AD, the pattern to activation with formix stimulation in the patients and the response latencies as seen with the EEG analysis were similar to that previously reported in applicant's cognitively intact patient that also received hypothalamic stimulation and had enhancement in his memory function (Hamani et al., 2008). As in this initial patient and now in the 6 AD patients, applicant's leading hypothesis is that the stimulation produces activation of the axons of the formix. This in turn leads activation of the immediate downstream structures and subsequently to the polysynaptically connected secondary structures including the default mode network. The increasing latency to activation of the cingulate and parietal area versus the hippocampus seen using sLORETA is consistent with this notion. The reason for postulating the formix and perhaps the mamillothalamic tract over other structures, especially hypothalamic nuclei and particularly the mammilary bodies is that first, axons are more sensitive than neuronal cell bodies to the effects of electrical stimulation (Ranck 1975) and second, applicant has seen that the acute experimental effects of stimulation are produced at a similar current threshold along the 4 electrode contacts that lie at various points along the vertical axis of the formix. Because the effects were mediated by the mammilary bodies, one would predict lower threshold at the deepest contact followed by increasing threshold at each of the more dorsal contacts. The predominantly ipsilateral physiological effects of stimulation seen with sLORETA (FIG. 3) and the lateralized specialization of memory with the right side preferentially mediating spatial memory and the left verbal memory speaks for the need for bilateral stimulation in widespread disorders like AD.

### Discussion—Other Supporting Observations

The findings of activation of memory circuits and improvement in glucose metabolism and the possible effect on clinical outcome mirror recent findings of DBS for Parkinson's disease (PD) and for depression. In PD, STN and globus pallidus and DBS is associated with activation of brain areas involved in the planning and initiation of movement (Davis et al., 1997) while in patients with severe depression, DBS of the subcallosal cingulate gyrus reversed the abnormalities in resting glucose metabolism across brain regions implicated in depression (Mayberg et al., 2005). In another phase 1 trial, NGF gene therapy for AD (Tuszynski et al, 2005) showed interval increases in brain metabolism and a similar potential clinical benefit (an annual decline in the ADAS-cog/11 of 6.2 points in the 8 patients receiving NGF gene therapy versus the 4.2 point decline in the DBS patients reported here). Applicant has preliminary evidence in laboratory rodents that electrical stimulation of the Papez circuit (of which the formix and hippocampus are part) using homologous parameters can drive neurotrophin expression and enhance neurogenesis in the hippocampus (Toda et al, 2008). Recent work suggests that enhancing the delivery of the neurotrophin BDNF in animal models of AD may improve reverse synaptic loss and improve cognitive function (Nagahara et al., 2008). Whether DBS regulates neurotrophin expression and neurogenesis in humans and whether this occurs in the diseased hippocampus of patients with AD is not known. The availability of animal models of AD will facilitate the examination of these questions.

### Discussion—Limitations

Applicant has shown that in this small group of patients, the procedure is well tolerated and produces biological effects with respect to activating the target circuits and reversing some of the metabolic abnormalities in glucose utilization.

### Discussion—Conclusions

Some 4.5 million Americans suffer with Alzheimer's disease and these numbers are expected to nearly triple by the year 2050 (Hebert et al., 2000). Approved treatments are directed at modulating neurotransmission in general and are not regionally targeted or specific. There is major dysfunction in cognitive and memory circuits in AD. As shown here, DBS offers the possibility of modulating these specific brain circuits in an adjustable and reversible fashion and it appears that this approach can be safe.

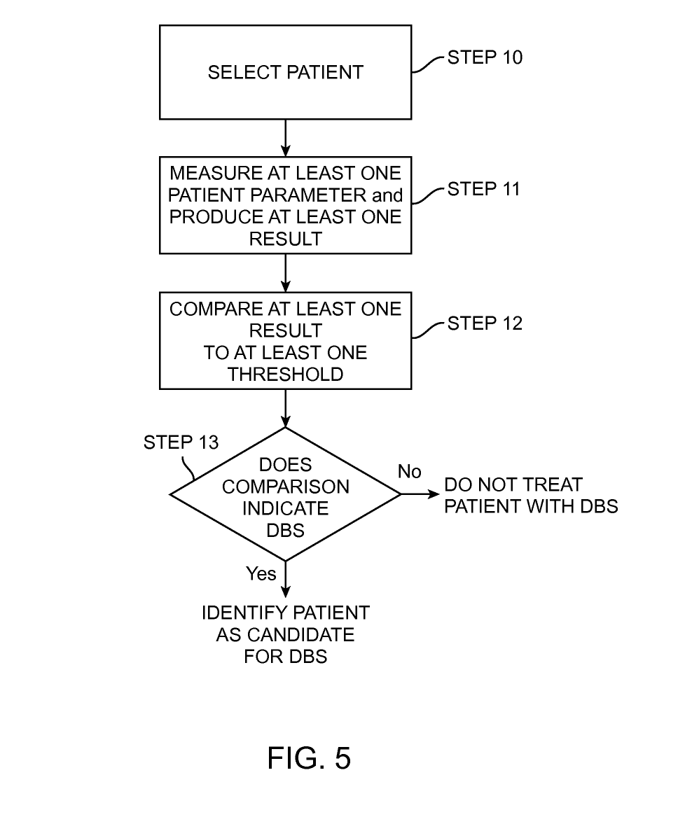

Referring now to FIG. 5, a flow chart of a method of screening a patient for deep brain stimulation is illustrated. In STEP 10, a patient is selected for screening. The patient is typically a patient who has been diagnosed with Alzheimer's disease, such as when the diagnosis has been performed within two years. The patient is typically between forty and eighty years old, such as a patient between fifty-five and eighty years old, and has a genetic form of Alzheimer's disease. The patient may have taken cholinesterase inhibitor, such as for a time period of at least six months. Numerous other factors, singly or in combination, may be used to select a patient for screening, such factors including but not limited to: diagnosis of being an Apo E4 allele carrier; diagnosis of mild cognitive impairment (MCI); presence of damage to the hippocampus such as due to anoxia, epilepsy or depression. Diffusion tensor imaging is an MRI technique that can be used to reveal abnormalities in white matter fiber structure and provide models of brain connectivity. In some embodiments, the patient can be identified for treatment based on a confirmation of reduced integrity of white matter tracts innervating limbic structures such as the formix, such as can be determined by fractional anisotropy maps using diffusion tensor imaging. Patients may be eliminated from screening due to one or more factors, such as a pre-existing structural brain abnormality including but not limited to: a tumor; an infarction; an intracranial hematoma; and combinations of these.

The patient may be screened to treat Alzheimer's disease and/or another neurological disease or disorder, such as to treat a cognitive function such as a memory function. The patient may be screened to treat memory impairment; improve memory function; reverse synaptic loss; treat cognitive function loss; improve cognitive function; and/or reduce degradation of cognitive function. The patient may be screened to promote neurogenesis in the hippocampus; drive neurotrophin expression; regulate biomarkers related to Alzheimer's disease such as abeta, tau, and phosphor tau; regulate BDNF expression; increase neurotransmitter release such as release of acetylcholine; and/or improve glucose utilization in the temporal or parietal lobes.

In STEP 11, one or more patient parameters are measured producing one or more results. These one or more results are used in the patient screening process in subsequent steps. Results may be data or other information directly representing the level or other status of a patient parameter, or may be data that is processed such as via one or more mathematical algorithms, hereinafter collectively referred to as result or results. The one or more patient parameters may be selected from the group consisting of: Mini-Mental State Examination (MMSE) level; Alzheimer's Disease Assessment Scale-Cognitive Subscale level such as ADAS-cog/11; a word recall test such as Item 1 of ADAS-cog; Clinical Dementia Rating-Sum of Boxes (CDR) level; Alzheimer's Disease Study Consortium-Activities of Daily Living level; Clinicians Interview-Based Impression of Change Plus Caregiver Input (CIBIC-plus) level; Neuropsychiatric Inventory (NPI) level; Electro Encephalography (EEG) signal, level or result of EEG signal analysis; PET image data or data analysis; FMRI image data or data analysis; MRI image data or data analysis such as hippocampal volume; diffusion tensor imaging data such as data including an assessment of the fractional anisotropy of the formix; and combinations of these.

In STEP 12 and STEP 13, the one or more results collected in STEP 11 are compared to one or more thresholds. In one embodiment, a single result is compared to a single threshold. In another embodiment, multiple results are processed and compared to one or more thresholds. In yet another embodiment, multiple results are compared individually or in combination to one or more thresholds. The one or more thresholds may comprise a maximum level, a minimum level, or an acceptable range of values within which the results should reside. Comparison of the results to the one or more thresholds will indicate if the patient has passed the screen, and is thus applicable for the deep brain stimulation of the present invention. In some embodiments, the results correlate to a level of progression of a cognitive disorder such as Alzheimer's disease. In these embodiments, the patient can be identified as a suitable candidate for the stimulation therapy of the present invention if these results indicate the severity of the patient's cognitive disorder is below a threshold (i.e. not too severe). For example, the patient may be identified as a suitable candidate when the results of an MMSE test, an ADAS-cog test, a word recall test and/or other test does not exceed a threshold (above or below as appropriate), such as when exceeding the threshold correlates to a level a disease progression that is not necessarily appropriate for the stimulation systems, methods and devices described herein.

The one or more collected results may include an MMSE score. In a particular embodiment, the at least one threshold is 20 and the patient is a candidate for DBS if the MMSE score is greater than or equal to 20. The at least one threshold may include a first threshold and a second threshold. In another embodiment, the patient is a candidate for DBS if the MMSE score is greater than or equal to a first threshold of 20, and less than or equal to a second threshold of 29, or less than or equal to a second threshold of 24. In some embodiments, the patient is a candidate for DBS if the ADAS-cog/11 score is greater than or equal to 12 (i.e. severity above a first threshold) and less than or equal to 24 (i.e. severity below a second threshold). In some embodiments, the patient is a candidate if a word recollection test exceeds a threshold, such as minimum threshold comprising the number of words the patient was unable to remember. In these embodiments, the threshold can be 4 out of 10 words not remembered (i.e. the threshold is exceeded if the patient can't remember between 4 and 10 words out of an original 10 words tested, such as when the test comprising the ADAS-cog item 1 test).

The at least one result may include a first result comprising an MMSE score and a second result comprising an ADAS-cog score. The at least one threshold may include a first threshold and a second threshold. In a particular embodiment, the patient is a candidate for DBS if the MMSE score is greater than or equal to a first threshold of 20, and the ADAS-cog/11 score is less than or equal to a second threshold of 24.

The at least one result may comprise an ADAS-cog Score. In a particular embodiment, the patient is a candidate for DBS if the ADAS-cog/11 score is less than or equal to a threshold of 24, or less than or equal to threshold of 20.

The at least one result may comprise a CDR score. In a particular embodiment, the patient is a candidate for DBS if the CDR score is less than or equal to a threshold, and when the threshold includes multiple values, such as a first value of 0.5 and a second value of 1.0.

The at least one result may comprise data obtained in a PET scan, such as a PET scan producing glucose utilization data. In a particular embodiment, the at least one result is Pittsburgh compound (PiB) data.

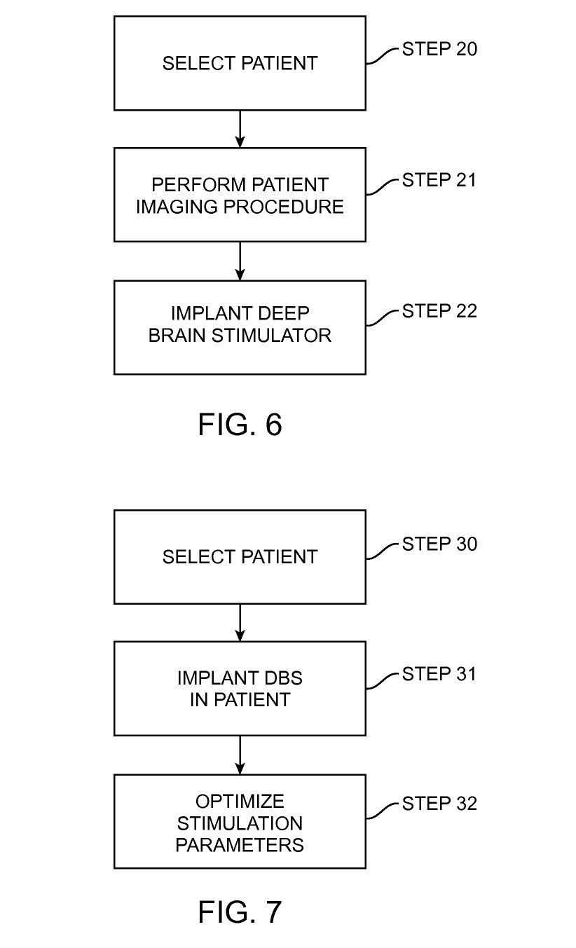

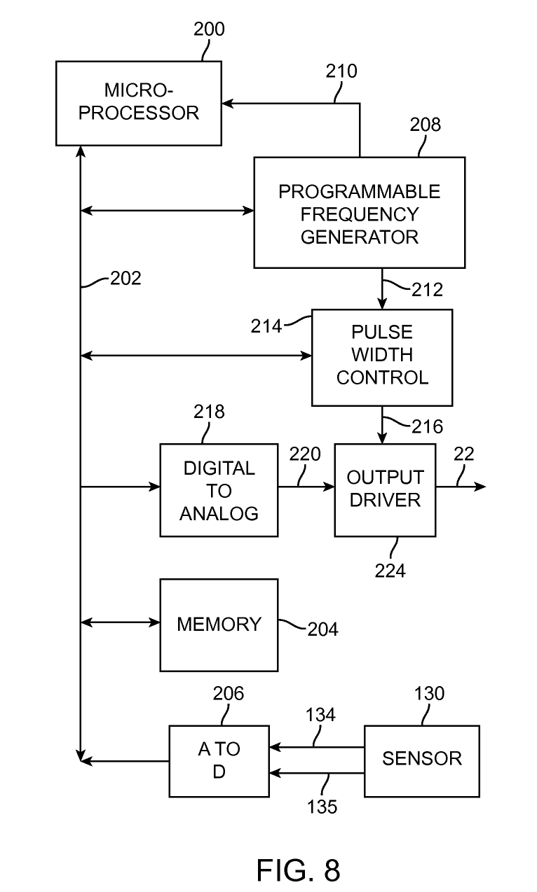

Once the patient has been identified as a candidate for DBS therapy, implantation of a DBS device, such as the DBS device described in reference to FIG. 8 herebelow, may be scheduled and performed. A preferred method of DBS implantation is described in reference to FIG. 6 immediately herebelow.

Referring now to FIG. 6, a flow chart of a method of implanting a deep brain stimulator is illustrated. In STEP 20, a patient is selected for implantation. In one method for implanting a deep brain stimulator, the patient is screened for candidacy as described in reference to FIG. 5 hereabove. In STEP 21, at least one imaging procedure is performed on the patient, collecting at least one patient image. In some embodiments, the imaging procedure is an MRI procedure performed to identify the formix of the patient. Alternatively or additionally, different patient imaging procedures can be used including imaging procedures selected from the group consisting of x-ray; ultrasound imaging; fMRI; PET scan; and combinations of these. Multiple imaging procedures may be performed, such as similar imaging procedures performed at different times, or different imaging procedures performed at the same or different times. In one embodiment, a first imaging procedure is performed at least 7 days prior to a second imaging procedure. In another preferred embodiment, a first imaging procedure is an MRI procedure and a second imaging procedure is selected from the group consisting of: a second MRI procedure; an x-ray; an ultrasound imaging procedure; an fMRI; a PET scan; and combinations of these. Multiple patient images, collected in one or more similar or dissimilar imaging procedures, can be collected. These images may be used in combination, in comparison, or both. In a particular embodiment, the two procedures are performed at different times and one or more patient parameters are compared, such as parameters selected from the group consisting of: brain size; brain shape; brain thickness; and combinations of these.

In STEP 21, the deep brain stimulator is implanted, such as the deep brain stimulator described in reference to FIG. 8 herebelow, including implanting at least one stimulation element of the deep brain stimulator in the patient. The at least one stimulation element is positioned in the brain of the patient, based on the at least one patient image. The at least one stimulation element may be placed via a visual analysis of the at least one image, and/or one or more mathematical or other computational analysis or analyses of the patient image. In a preferred embodiment, the at least one stimulation element is positioned in the Papez circuit of the patient's brain. In another embodiment, the at least one stimulation element, such as a stimulation element comprising at least two electrodes, is positioned approximately 2 mm anterior and parallel to the vertical portion of the formix within the hypothalamus. The stimulation element may comprise at least one electrode. In a particular embodiment, the ventral-most electrode is positioned approximately 2 mm above the dorsal surface of the optic tract, approximately 5 mm from the midline. Proper positioning of the stimulation element may be confirmed after placement, such as with a subsequent MRI image.

The stimulation element may comprise an electrical stimulation element such as an electrode or a magnet such as an electromagnet. Alternatively or additionally, the stimulation element may comprise an optical stimulation element, such as a visible light element; an infrared light element; and combinations of these. Alternatively or additionally, the stimulation element may comprise a chemical stimulation element, such as a drug delivery assembly. The drug delivery assembly may be configured to deliver one or more of: biologically active molecules; neurotransmitters; and neurotrophic factors. The stimulation element may deliver one or more drugs or pharmaceutical agents, and delivery rate or drug concentration may be determined based on patient tolerance, such as a tolerance determined in a titration procedure. In a particular embodiment, the stimulation element is constructed and arranged to deliver a cholinesterase inhibitor. In another particular embodiment, an electrode and a second stimulation element is included. The second stimulation element may comprise an element selected from the group consisting of: a second electrode; a magnet; an optical element; a chemical or other agent delivery assembly; and combinations of these.

During or after the implantation of the DBS device, one or more stimulating elements may be repositioned. This repositioning may be based on maximizing a patient parameter, such as maximizing recalled memory or memories. Alternatively or additionally, the repositioning may be based on minimizing a patient parameter, such as to minimize chest pain; undesired EKG signal or signals; undesired EEG signal or signals; labored breathing; twitching; and combinations of these. Alternatively or additionally, the repositioning may be based on minimizing a neurological condition of the patient, such as a level of one or more of: paranoia; psychosis; anxiety; or confusion.

During or after the implantation of the DBS device, a decision may be made to explant the implanted or partially implanted device. The implantation procedure may include a calibration or titration procedure, such as procedures which optimize or otherwise modify stimulation parameters such as parameters selected from the group consisting of: electromagnetic energy delivery such as voltage or current delivered; light delivery such as wavelength or magnitude of light delivered; chemical parameters such as concentration of chemical delivered or rate of chemical delivery; and combinations of these. If successful calibration or titration cannot be achieved, the DBS device may be removed and the procedure abandoned. Alternatively, if a particular patient event occurs, such as a patient adverse event, the DBS device may be explanted. Typical patient events causing explantation may include but are not limited to: chest pain; labored breathing; twitching; unacceptable EKG signal or combination of signals; unacceptable EEG signal or combination of signals; and combinations of these. Alternatively or additionally, typical patient events causing explantation may be an unacceptable neurological state such as an unacceptable level of one or more of: paranoia; psychosis; anxiety; or confusion.

Referring now to FIG. 7, a method of optimizing stimulation parameters of a deep brain stimulator of the present invention is illustrated. In STEP 30, a patient is selected for implantation, such as via the screening method described in reference to FIG. 5. In STEP 31, a deep brain stimulator is implanted such as the deep brain stimulator described in reference to FIG. 8 herebelow, per the implantation method described in reference to FIG. 6 hereabove.

In STEP 32, one or more stimulation parameters are optimized. Stimulation parameters optimized can relate to energy delivery amounts and forms. In a particular embodiment, electrical energy is delivered by one or more electrodes and the parameter modified is selected from the group consisting of: voltage; current; frequency; duty cycle; and combinations thereof. During or after the implantation, one or more stimulation parameters may be optimized or otherwise modified. The modification may be determined after a level of patient discomfort is achieved, such as discomfort including one or more of: sweating; hallucinations; visual sensation; or tingling. Stimulation parameters may be modified based on one or more of: EEG recordings; magnetoencephalography recordings; or other patient physiologic recording. Stimulation parameters may be modified after analysis of a PET scan, such as a measurement of blood flow and/or fluorodeoxyglucose (FDG) data. Stimulation parameters may be modified based on the results of an acute memory test, such as an acute memory test performed at varying stimulation settings. The acute memory test can include asking the patient to recall words and/or images.

Referring now to FIG. 8, a schematic of a deep brain stimulator of the present invention is illustrated. Stimulation device 16 delivers a stimulus pulse frequency that is controlled by programming a value to a programmable frequency generator 208 using bus 202. The programmable frequency generator provides an interrupt signal to microprocessor 200 through an interrupt line 210 when each stimulus pulse is to be generated. The frequency generator 208 may be implemented by model CDP1878 sold by Harris Corporation. The amplitude for each stimulus pulse is programmed to a digital to analog converter 218 using bus 202. The analog output is conveyed through a conductor 220 to an output driver circuit 224 to control stimulus amplitude.

Microprocessor 200 also programs a pulse width control module 214 using bus 202. The pulse width control 214 provides an enabling pulse of duration equal to the pulse width via a conductor 216. Pulses with the selected characteristics are then delivered from device 16 through cable 22 to the Papez circuit and/or other regions of the brain. At the time the stimulation device 16 is implanted, the clinician may program certain key parameters into the memory of the implanted device such as via telemetry. These parameters may be updated subsequently as needed.

Deep brain stimulation electrodes, such as Medtronic model 3387; Medtronic, Minneapolis, Minn., may be bilaterally implanted such that the tips of the electrodes are positioned in a region where cells could still be recorded during micro-recording mapping. Energy is typically applied at a frequency of 20 to 200 Hz, such as at a frequency of 130 Hz. Energy is typically delivered at a voltage between 1.0 and 10.0 volts, such as at a voltage between 3.0 and 3.5 volts. In a particular embodiment, the voltage applied is less than 7.0 volts. Energy delivery may be given in a series of on and off times, such as with an on-time of approximately 45 μseconds to 450 μseconds, such as with an on time of 90 μseconds.

The embodiments of the present invention shown above are typically open-loop systems. The microcomputer algorithm programmed by the clinician sets the stimulation parameters of signal generator 16. This algorithm may change the parameter values over time but does so independent of any changes in symptoms the patient may be experiencing. Alternatively, a closed-loop system discussed below which incorporates a sensor 130 to provide feedback could be used to provide enhanced results. Sensor 130 can be used with a closed loop feedback system in order to automatically determine the level of electrical stimulation necessary to achieve the desired level of improved cognitive function. In a closed-loop embodiment, microprocessor 200 executes an algorithm in order to provide stimulation with closed loop feedback control. Such an algorithm may analyze a sensed signal and deliver the electrical of chemical treatment therapy based on the sensed signal falling within or outside predetermined values or windows, for example, for BDNF and other neurotrophins (e.g., NGF, CNTF, FGF EGF, NT-3) and corticosteroids.

For example, in one embodiment, the patient may engage in a specified cognitive task, wherein the system measures one or more characteristics to determine if the sensed levels are at expected thresholds. If one or more of the sensed characteristics are outside a predetermined threshold, the system may initiate and/or regulate the treatment therapy to thereby enhance cognitive function.

In one embodiment, the system may be continuously providing closed-loop feedback control. In another embodiment, the system may operate in closed-loop feedback control based on a time of day (e.g., during hours that the patient is awake) or based on a cognitive task (e.g., when the patient is working). In yet another embodiment, the system may be switchable between open-loop and closed-loop by operator control.

In another embodiment, the stimulation or drug delivery could be applied before, after and/or during the performance of a memory, cognitive or motor task learning task to facilitate the acquisition of learning or consolidation of the task and in so doing, accelerate the rate of memory acquisition and learning and enhance its magnitude. For example, the stimulation or drug or other chemical delivery may be provided before, during, or after periods when the patient is learning a new language or playing a new instrument. Such therapy may be useful during the encoding, consolidation and/or retrieval phases of memory. The neuromodulation intervention, brain stimulation or drug delivery could occur before, after or simultaneously to the memory, cognitive of motor skill task.

In another embodiment, therapy may be provided in relation to a learning task. For example, the stimulation or drug delivery could be applied before, after and/or during the performance of a memory, cognitive or motor task to facilitate the acquisition of learning or consolidation of the task. In so doing, the rate of memory acquisition and learning may be accelerated and enhanced in magnitude. For example, the stimulation or drug delivery may be provided before, during, or after periods when the patient is learning a new language or playing a new instrument. Such therapy may be useful during the encoding, consolidation and/or retrieval phases of memory. The neuromodulation intervention, brain stimulation or drug delivery could occur before, after or simultaneously to the memory, cognitive of motor skill task.

In another aspect of the invention, treatment therapy may be utilized to enhance neurogenesis as a method of improving cognitive function. Techniques for enhancing neurogenesis through treatment therapy are disclosed in patent applications “Cognitive Function Within A Human Brain”, U.S. Ser. No. 11/303,293; “Inducing Neurogenesis Within A Human Brain”, U.S. Ser. No. 11/303,292; “Regulation of Neurotrophins”, U.S. Ser. No. 11/303,619; “Method Of Treating Cognitive Disorders Using Neuromodulation”, U.S. Ser. No. 11/364,977; each assigned to applicant and incorporated herein by reference in its entirety.

Referring back to FIG. 8, the system may optionally utilize closed-loop feedback control having an analog to digital converter 206 coupled to sensor 130. Output of the A-to-D converter 206 is connected to microprocessor 200 through peripheral bus 202 including address, data and control lines. Microprocessor 200 processes sensor data in different ways depending on the type of transducer in use and regulates delivery, via a control algorithm, of electrical stimulation and/or drug delivery based on the sensed signal. For example, when the signal on sensor 130 exceeds a level programmed by the clinician and stored in a memory 204, increasing amounts of stimulation may be applied through an output driver 224. In the case of electrical stimulation, a parameter of the stimulation may be adjusted such as amplitude, pulse width and/or frequency.

Parameters which could be sensed include the activity of single neurons as detected with microelectrode recording techniques, local field potentials, event related potentials, for example in response to a memory task or sensory stimulus and electroencephalogram or electrocorticogram. For example, U.S. Pat. No. 6,227,203 provides examples of various types of sensors that may be used to detect a symptom or a condition of a cognitive disorder and responsively generate a neurological signal. In an embodiment, a neurochemical characteristic of the cognitive function may be sensed, additionally or alternatively. For example, sensing of local levels of neurotransmitters (glutamate, GABA, Aspartate), local pH or ion concentration, lactate levels, local cerebral blood flow, glucose utilization or oxygen extraction may also be used as the input component of a closed loop system. These measures could be taken at rest or in response to a specific memory or cognitive task or in response to a specific sensory or motor stimulus. In another embodiment, an electro-physiological characteristic of the cognitive function may be sensed. The information contained within the neuronal firing spike train, including spike amplitude, frequency of action potentials, signal to noise ratio, the spatial and temporal features and the pattern of neuronal firing, oscillation behavior and inter-neuronal correlated activity could be used to deliver therapies on a contingency basis in a closed loop system. Moreover, treatment therapy delivered may be immediate or delayed, diurnal, constant or intermittent depending on contingencies as defined by the closed loop system.

While the systems, methods and devices of the present invention have mainly been described in reference to treatment for Alzheimer's disease and MCI, numerous other neurological conditions and disorders may be applicable including but not limited to: chronic pain; dystonia; Parkinson's disease; Huntington's disease; depression; other neurological disorders and diseases; and combinations of these.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

What is claimed is:

1. A method for screening patients prior to deep brain stimulation therapy to treat cognitive function, said method comprising:
measuring at least one patient parameter to generate at least a first result;
comparing the first result to a first threshold;
identifying the patient as a candidate for deep brain stimulation therapy based on said comparison of the first result to the first threshold.

2. The method of claim 1 wherein the cognitive function treated comprises a memory function.

3. The method of claim 1 wherein the patient has been diagnosed with a disease or disorder selected from the group consisting of: probable Alzheimer's disease; a genetic form of Alzheimer's disease; mild cognitive impairment; hippocampal damage such as hippocampal damage due to Alzheimer's disease, anoxia, epilepsy or depression; and combinations thereof.

4. The method of claim 3 wherein the diagnosis was performed within the prior two years.

5. The method of claim 1 wherein the patient comprises a patient between forty and eighty years of age.

6. The method of claim 1 wherein the patient has been diagnosed as an Apo E4 allele carrier.

7. The method of claim 1 wherein the patient has reduced integrity of white matter tracts innervating limbic structures such as the formix as determined by fractional anisotropy maps using diffusion tensor imaging.

8. The method of claim 1 wherein the deep brain stimulation therapy achieves at least one of: treats memory impairment; improves memory function; treats cognitive function loss; reverses synaptic loss; improves cognitive function; reduces degradation of cognitive function; promotes neurogenesis in the hippocampus of the patient's brain; drives neurotrophin expression; regulates one or more biomarkers related to Alzheimer's disease such as abeta, tau, and/or phosphorylated tau; regulates BDNF expression; increases neurotransmitter release such as acetylcholine; or improves glucose utilization in the temporal lobe, the parietal lobe or both lobes of the patient's brain.

9. The method of claim 1 wherein the at least one patient parameter is selected from the group consisting of: Mini-Mental State Examination (MMSE) level; Alzheimer's Disease Assessment Scale-Cognitive Subscale level; Clinical Dementia Rating-Sum of Boxes (CDR) level; Alzheimer's Disease Study Consortium—Activities of Daily Living level; Clinicians Interview-Based Impression of Change Plus Caregiver Input (CIBIC-plus) level; Neuropsychiatric Inventory (NPI) level; Electro Encephalography (EEG) signal, level or result of EEG signal analysis; PET image data or data analysis; FMRI image data or data analysis; MRI image data or data analysis such as hippocampal volume, diffusion tensor imaging data such as data including an assessment of the fractional anisotropy of the formix; and combinations thereof.

10. The method of claim 1 wherein the first result comprises an MMSE score, wherein the first threshold comprises an MMSE value of 20, and wherein the patient is a candidate for DBS if said first result is greater than or equal to the first threshold.

11. The method of claim 10 further comprising comparing the first result to a second threshold, said second threshold comprising an MMSE value of 29, wherein the patient is a candidate for DBS if said first result is less than or equal to said second threshold.

12. The method of claim 10 further comprising generating a second result and comparing said second result to a second threshold, said second result comprising an ADAS-cog/11 score, said second threshold comprising an ADAS-cog/11 score value of 24, and wherein the patient is a candidate for DBS if said second result is less than or equal to said second threshold.

13. The method of claim 1 wherein the first result comprises an ADAS-cog/11 score, wherein the first threshold comprises an ADAS-cog/11 score value of 24, and wherein the patient is a candidate for DBS if said first result is less than or equal to the first threshold.

14. The method of claim 13 further comprising generating a second result and comparing the second to result to a second threshold, said second result comprising an MMSE, said second threshold comprising an MMSE value of 20, and wherein the patient is a candidate for DBS if said second result is greater than or equal to said second threshold.

15. The method of claim 1 wherein the first result comprises an ADAS-cog Item 1 score, wherein the first threshold comprises a score of 4, and wherein the patient is a candidate for DBS if the first result is greater than or equal to the first threshold.

16. The method of claim 1 wherein the first result comprises a CDR score, wherein the first threshold comprises a set of values including 0.5 and 1.0, and wherein the patient is a candidate for DBS if said first result is included in the threshold set of values.

17. The method of claim 1 wherein the first result comprises data obtained in a PET scan.

18. The method of claim 1 further comprising implanting a deep brain stimulator in the patient.

19. The method of claim 18 further comprising performing an MRI procedure prior to or during said stimulator implantation to produce at least one MRI image and wherein said stimulator comprises a stimulating portion that is implanted relative to a formix target identified on the at least one MRI image.

20. The method of claim 18 wherein implanting a deep brain stimulator comprises implanting one or more electrodes in a location selected from the group consisting of: in the Papez Circuit of the patient's brain; approximately 2 mm anterior and parallel to the vertical portion of the formix; in the optic tract such that the ventral most contact is 2 mm above the dorsal surface of the optic tract, approximately 5 mm from the midline; and combinations thereof.

21. The method of claim 18 further comprising delivering energy at a frequency between 20 and 200 Hz.

22. The method of claim 18 further comprising delivering energy at a voltage between 1.0V and 10.0V.

23. The method of claim 18 further comprising delivering energy in multiple pulses of 45-450 μsecond duration.

24. The method of claim 18 further comprising performing an optimization of stimulation parameters procedure.

25. The method of claim 24 wherein the optimization is performed by determining a maximum level of patient discomfort wherein the patient discomfort is selected from the group consisting of: sweating; hallucinations; visual sensations; tingling; and combinations thereof.

26. The method of claim 24 wherein said optimization procedure is performed at least one of during or after the stimulator implantation surgery.

27. The method of claim 24 further comprising recording at least one of EEG data or magnetoencephalography data, wherein the optimization procedure is based on said data.

28. The method of claim 24 further comprising performing a PET scan wherein data collected during the PET scan is selected from the group comprising: blood flow; FDG data; and combinations thereof, wherein said optimization procedure is based on the collected data.

29. The method of claim 24 further comprising performing an acute test of memory at varying stimulation settings, wherein the acute memory test comprises a patient recall of at least one of words or images and wherein the optimization procedure is based on results of the acute memory test.

30. The method of claim 18 wherein the implanting of the deep brain stimulator comprises implanting a stimulating element, wherein the method further comprises confirming the placement of the stimulating electrode using an imaging instrument.

31. The method of claim 18 further comprising delivering a drug or other agent to the patient wherein the drug or other agent comprises at least one cholinesterase inhibitor.

32. The method of claim 18 further comprising assessing a patient tolerance to at least one drug and delivering the at least one drug to the patient if the patient tolerance is within a clinically acceptable limit, wherein the at least one drug is a cholinesterase inhibitor.
